(19) United States
(12) Patent Application Publication
(10) Pub. No.: US 2025/0160796 A1
(43) Pub. Date: May 22, 2025

(54) ULTRASONIC DIAGNOSTIC APPARATUS, ULTRASONIC DIAGNOSTIC SYSTEM, AND STORAGE MEDIUM

(71) Applicant: Konica Minolta, Inc., Tokyo (JP)

(72) Inventor: Tetsuya TANIGUCHI, Tokyo (JP)

(21) Appl. No.: 18/934,875

(22) Filed: Nov. 1, 2024

(30) Foreign Application Priority Data
Nov. 17, 2023 (JP) 2023-195591

(51) Int. Cl. A61B 8/00; A61B 8/08

(52) U.S. Cl. CPC A61B 8/466; A61B 8/4427; A61B 8/4472; A61B 8/462; A61B 8/483; A61B 8/56

(57) ABSTRACT

An ultrasonic diagnostic apparatus includes an ultrasonic probe that transmits an ultrasonic wave to a subject and receives an ultrasonic image signal reflected in the subject, a communicator that transmits image data based on the ultrasonic image signal of the subject received by the ultrasonic probe to an information processing apparatus, a hardware processor that acquires diagnostic information obtained by the information processing apparatus constructing, using the image data, three-dimensional image data and performing a predetermined process on the three-dimensional image data, and a display that displays the acquired diagnostic information.

### CROSS-REFERENCE TO RELATED APPLICATIONS

The entire disclosure of Japanese Patent Application No. 2023-195591 filed on Nov. 17, 2023 is incorporated herein by reference in its entirety.

### BACKGROUND OF THE INVENTION

### Technical Field

The present invention relates to an ultrasonic diagnostic apparatus, an ultrasonic diagnostic system, and a storage medium.

### Description of Related Art

In recent years, at the site of home nursing, remote diagnosis (online diagnosis) by a doctor or the like using an information terminal such as a smartphone, a personal computer or a tablet has widely spread. In remote diagnosis, there are increasing cases where, instead of a doctor, paramedical staff such as a nurse performs a treatment action such as an injection or an intravenous drip on the basis of an instruction from a doctor at a remote location. At such a site, an ultrasonic diagnostic apparatus that is easier for a nurse or the like to use and understand is more desirable than a highly specialized ultrasonic diagnostic apparatus. Examples thereof include an ultrasonic diagnostic apparatus capable of three-dimensionally and stereoscopically displaying complicated tissues which is difficult to understand with a tomographic image, and a small ultrasonic apparatus which is easy to carry.

In Japanese Unexamined Patent Publication No. 2013-212184, there is disclosed an ultrasonic diagnostic apparatus including a display provided on one surface of a housing and an ultrasonic probe section provided on the other surface of the housing. A tomographic image of soft tissues of a living body is displayed on the touch screen's display. According to the ultrasonic diagnostic apparatus disclosed in Japanese Unexamined Patent Publication No. 2013-212184, reduction in size can be achieved.

### SUMMARY OF THE INVENTION

Incidentally, in order to perform a complicated process such as three-dimensional display in an ultrasonic image, a large and expensive apparatus equipped with a processor capable of high-speed processing is required. The ultrasonic diagnostic apparatus disclosed in Japanese Unexamined Patent Publication No. 2013-212184 is capable of processing a simple two-dimensional image, but its processing performance is insufficient to execute complicated rendering of a three-dimensional image or the like in real time. Mounting a high-speed processor on/in the housing to improve the processing performance requires increase in size or the like of the housing. Further, in order to display a three-dimensional image with high definition, a large display is also required.

In view of these, an object of the present invention is to provide an ultrasonic diagnostic apparatus, an ultrasonic diagnostic system and a storage medium storing a program capable of displaying simplified information easily understood by a user such as a nurse by making use of information on a three-dimensional image while reduction in size of the ultrasonic diagnostic apparatus is achieved by the ultrasonic diagnostic apparatus not displaying the three-dimensional image.

To achieve at least one of the abovementioned objects, according to an aspect of the present invention, an ultrasonic diagnostic apparatus reflecting one aspect of the present invention includes:
an ultrasonic probe that transmits an ultrasonic wave to a subject and receives an ultrasonic image signal reflected in the subject;
a communicator that transmits image data based on the ultrasonic image signal of the subject received by the ultrasonic probe to an information processing apparatus;
a hardware processor that acquires diagnostic information obtained by the information processing apparatus constructing, using the image data, three-dimensional image data and performing a predetermined process on the three-dimensional image data; and
a display that displays the acquired diagnostic information.

According to an aspect of the present invention, an ultrasonic diagnostic system reflecting one aspect of the present invention includes:
an ultrasonic diagnostic apparatus that transmits an ultrasonic wave to a subject and acquires image data corresponding to an ultrasonic image signal reflected in the subject, and transmits the image data; and
an information processing apparatus that constructs, using the image data transmitted from the ultrasonic diagnostic apparatus, three-dimensional image data, acquires diagnostic information by performing a predetermined process on the three-dimensional image data, and transmits the diagnostic information,
wherein the ultrasonic diagnostic apparatus receives the diagnostic information transmitted from the information processing apparatus, and displays the diagnostic information on a display thereof.

According to an aspect of the present invention, a non-transitory computer-readable storage medium reflecting one aspect of the present invention stores a program causing a computer to perform:
transmitting, to an information processing apparatus, image data based on an ultrasonic image signal obtained by transmission and reception of an ultrasonic wave to and from a subject;
acquiring diagnostic information obtained by the information processing apparatus constructing, using the image data, three-dimensional image data and performing a predetermined process on the three-dimensional image data; and
outputting the acquired diagnostic information.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram showing an example of a schematic configuration of an ultrasonic diagnostic system according to the present embodiment;

FIG. 2 is a block diagram of an ultrasonic diagnostic apparatus according to the present embodiment;

FIG. 3 is a block diagram of an information terminal according to the present embodiment;

FIG. 4 is a diagram showing an example of the configuration of a transmission/reception condition table stored in a storage section;

FIG. 5 is a sequence diagram illustrating a flow of information and data between the ultrasonic diagnostic apparatus and the information terminal according to the present embodiment;

FIG. 6 is a flowchart illustrating an example of the operation of the ultrasonic diagnostic apparatus when a urine volume measurement mode is selected as a measurement mode according to the present embodiment;

FIG. 7A is a diagram illustrating an example of diagnostic information displayed on a display part when the urine volume measurement mode ends normally;

FIG. 7B is a diagram illustrating an example of the diagnostic information displayed on the display part when the urine volume measurement mode does not end normally;

FIG. 8 is a flowchart illustrating an example of the operation of the terminal apparatus when the urine volume measurement mode is selected as the measurement mode according to the present embodiment;

FIG. 9 is a flowchart illustrating an example of the operation of the ultrasonic diagnostic apparatus when an inferior aorta diameter measurement mode is selected as the measurement mode according to the present embodiment;

FIG. 10A is a diagram illustrating an example of the diagnostic information displayed on the display part when the inferior aorta diameter measurement mode ends normally;

FIG. 10B is a diagram illustrating an example of the diagnostic information displayed on the display part when the inferior aorta diameter measurement mode does not end normally.

FIG. 11 is a flowchart illustrating an example of the operation of the terminal apparatus when the inferior aorta diameter measurement mode is selected as the measurement mode according to the present embodiment;

FIG. 12 is a flowchart showing an example of the operation of the ultrasonic diagnostic apparatus when a stool hardness measurement mode is selected as the measurement mode according to the present embodiment;

FIG. 13A is a diagram showing an example of the diagnostic information displayed on the display part when the stool hardness measurement mode is normally executed;

FIG. 13B is a diagram illustrating an example of the diagnostic information displayed on the display part when no feces are detected in the stool hardness measurement mode;

FIG. 13C is a diagram illustrating an example of the diagnostic information displayed on the display part when no intestine is detected in the stool hardness measurement mode;

FIG. 14 is a flowchart illustrating an example of the operation of the terminal apparatus when the stool hardness measurement mode is selected as the measurement mode according to the present embodiment;

FIG. 15 is a flowchart illustrating an example of the operation of the ultrasonic diagnostic apparatus when a blood collection guide mode is selected as the measurement mode according to the present embodiment;

FIG. 16A is a diagram illustrating an example of the diagnostic information displayed on the display part when the blood collection guide mode ends normally;

FIG. 16B is a diagram illustrating an example of the diagnostic information displayed on the display part when the blood collection guide mode ends normally;

FIG. 16C is a diagram illustrating an example of the diagnostic information displayed on the display part when the blood collection guide mode ends normally;

FIG. 17 is a flowchart illustrating an example of the operation of the terminal apparatus when the blood collection guide mode is selected as the measurement mode according to the present embodiment;

FIG. 18 is a subroutine of a lumen structure determination process illustrated in FIG. 17;

FIG. 19 is a subroutine of a simple image generation process illustrated in FIG. 17;

FIG. 20A is a view for explaining a procedure for generating a simple image of nerves as a lumen region according to the present embodiment; and

FIG. 20B is a view for explaining the procedure for generating a simple image of nerves as a lumen region according to the present embodiment.

### DETAILED DESCRIPTION

Hereinafter, one or more embodiments of the present invention will be described with reference to the drawings. However, the scope of the invention is not limited to the disclosed embodiments.

### Example Configuration of Ultrasonic Diagnostic System 1

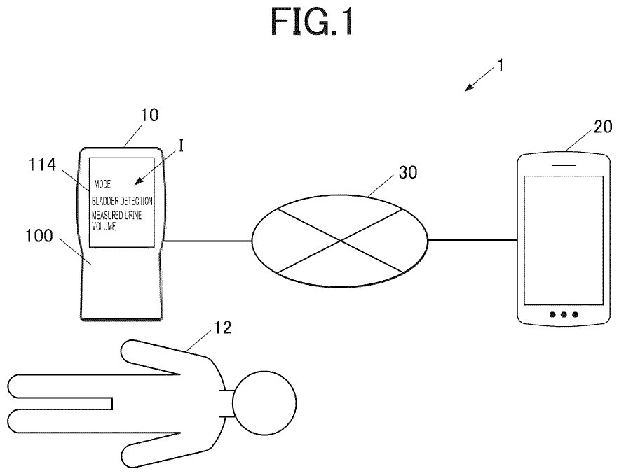

FIG. 1 shows an example of a schematic configuration of an ultrasonic diagnostic system 1 according to the present embodiment.

The ultrasonic diagnostic system 1 includes an ultrasonic diagnostic apparatus 10 and an information processing apparatus (terminal apparatus, information terminal) 20. The ultrasonic diagnostic apparatus 10 and the information processing apparatus 20 are communicably connected to each other via a network 30. Examples of the network 30 include a mobile communication network, the Internet, a WAN, and a LAN. WAN is an abbreviation for Wide Area Network. LAN is an abbreviation for Local Area Network. The network 30 may be a wired communication or a wireless communication.

The ultrasonic diagnostic apparatus 10 is used by a user such as a nurse at the site of home care, a medical facility such as a hospital, or the like. The ultrasonic diagnostic apparatus 10 images the shape, size, and the like of an organ or the like of a subject S by transmission and reception of ultrasonic waves. The ultrasonic diagnostic apparatus 10 receives diagnostic information I obtained by the information processing apparatus 20 numerically converting or simplifying three-dimensional image data and transmitted from the information processing apparatus 20, and displays the received diagnostic information I on the screen of a display part 114. In the present embodiment, the ultrasonic diagnostic apparatus 10 is reduced in size and weight by integrally configuring an ultrasonic probe (probe), a housing 100, and a display part 114 (display), which will be described later.

The information processing apparatus 20 is, for example, a smartphone, a tablet, a general-purpose personal computer, or a workstation. In the present embodiment, a smartphone is taken as an example of the information processing apparatus 20. The information processing apparatus 20 constructs a three-dimensional image using image data or the like of a tomographic image of the subject S transmitted from the ultrasonic diagnostic apparatus 10, and performs a predetermined process on the three-dimensional image, thereby generating the diagnostic information I that is numerically-converted or simplified three-dimensional image.

### Configuration Example of Ultrasonic Diagnostic Apparatus 10

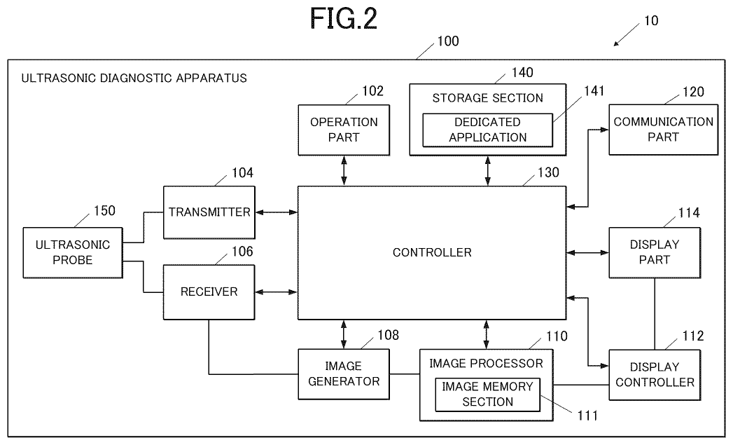

FIG. 2 is a block diagram of the ultrasonic diagnostic apparatus 10 according to the present embodiment. The ultrasonic diagnostic apparatus 10 includes a housing 100, an operation part 102, a transmitter 104, a receiver 106, an image generator 108, and an image processor 110. The ultrasonic diagnostic apparatus 10 further includes a display controller 112, a display part 114 (display), a communication part 120 (communicator), a controller 130 (hardware processor), a storage section 140, and an ultrasonic probe 150.

The housing 100 has, for example, a size for a user to hold. In the housing 100, constituent parts such as the above-described transmitter 104, communication part 120, controller 130, and ultrasonic probe 150 are mounted. On the surface of the housing 100, the operation part 102 and the display part 114 are provided. In this way, by configuring the ultrasonic diagnostic apparatus 10 in an integrated manner, the ultrasonic diagnostic apparatus 10 that is portable and that is reduced in size and weight is provided.

The operation part 102 receives various input operations from the user, converts the received input operations to electrical signals, and outputs the electrical signals to the controller 130. Specifically, the operation part 102 receives an input of selection of a measurement mode, power-on/off, or the like by the user. Examples of the measurement mode include a urine volume measurement mode, an inferior aorta diameter measurement mode, a stool hardness estimation mode, and a blood collection guide mode. The operation part 102 includes, for example, a switch, a button, and the like. The operation part 102 may be, for example, a touch screen integrally combined with a display. The operation part 102 may be, for example, a user interface such as a microphone that receives a voice input.

Under the control of the controller 130, the transmitter 104 supplies a drive signal, which is an electrical signal, to the ultrasonic probe 150. The transmitter 104 includes, for example, a clock generation circuit, a delay circuit, and a pulse generation circuit. The clock generation circuit generates a clock signal which determines a transmission timing and a transmission frequency of the drive signal. The delay circuit sets a delay time for each path provided in each transducer forming the ultrasonic probe 150, and delays transmission of the drive signal by the set delay time. The delay circuit converges transmission beams formed by transmission ultrasonic waves. The pulse generation circuit generates a pulse signal as a driving signal at a predetermined cycle. The transmitter 104 drives, for example, a continuous part of the transducers to generate transmission ultrasonic waves. The transmitter 104 performs scanning while shifting transducers to be driven in the azimuth direction each time transmission ultrasonic waves are generated.

Under the control of the controller 130, the receiver 106 receives, from the ultrasonic probe 150, reception signals that are ultrasonic image signals. The receiver 106 includes, for example, an amplifier, an A/D conversion circuit, and a phasing addition circuit. The amplifier amplifies the reception signal at a preset amplification factor for each path provided in the transducers forming the ultrasonic probe 150. The A/D conversion circuit performs analog-digital conversion on the amplified reception signal. The phasing addition circuit gives a delay time to the A/D converted reception signal for each path provided in each transducer to adjust the time phase, and adds these. The phasing addition circuit generates sound ray data (sound ray signals) by phasing addition. Note that the receiver 106 may include an amplifier for amplifying the reception signal.

The image generator 108 performs envelope detection, logarithmic compression, and the like on the sound ray data supplied from the receiver 106 under the control of the controller 130. The image generator 108 further adjusts the dynamic range and gain of the sound ray data and converts the brightness thereof, to generate, for example, B-mode image data. The B-mode image data represents the intensity of the reception signal by brightness, and is tomographic image information on tissues in the subject. Note that although the image generator 108 generates B-mode image data, there is no limitation, and another scanning mode may be used. Examples of the other scan mode (image mode) include an A mode, an M mode, and a scan mode using a Doppler method. Examples of the Doppler method include a color Doppler mode and a PWD. The B mode is an abbreviation for Brightness mode. The A mode is an abbreviation for Amplitude mode. The M mode is an abbreviation for Motion mode. The PWD is an abbreviation for Pulsed Wave Doppler.

The image processor 110 performs image processing on the B-mode image data output from the image generator 108 under the control of the controller 130. The image processor 110 performs image processing on the B-mode image data in accordance with various image parameters being set. The image processor 110 includes an image memory section 111 constituted by a semiconductor memory such as a DRAM. The DRAM is an abbreviation for Dynamic Random Access Memory. The image processor 110 stores the B-mode image data subjected to the image processing in the image memory section 111 in units of frames under the control of the controller 130. In the present embodiment, image data in units of frames may be referred to as ultrasonic image data or frame image data. Under the control of the controller 130, the image processor 110 sequentially outputs the image data generated as described above to the display controller 112.

The display controller 112 generates an image signal for display by performing coordinate conversion or the like on the received ultrasonic image data under the control of the controller 130. The display controller 112 outputs the generated image signal for display to the display part 114.

The display part 114 is, for example, a display apparatus such as a liquid crystal display or an organic EL display. The EL is an abbreviation for Electronic Luminescence. The liquid crystal display or the organic EL display is a flat panel or a bendable panel. The display part 114 displays a still image or the like corresponding to the image signal for display output from the display controller 112 on the display screen under the control of the controller 130. For example, the display part 114 displays the diagnostic information I, which is generated by the information processing apparatus 20 performing a predetermined process on a three-dimensional image, thereby numerically converting or simplifying the three-dimensional image.

The communication part 120 includes, for example, an NIC, a LAN adapter, and a communication module including a receiver and a transmitter. For example, the communication part 120 communicates various data such as ultrasonic image data, transmission/reception condition information, and diagnostic information I with the information processing apparatus 20 or the like connected via the network 30.

The controller 130 includes a processor such as a CPU, a memory such as a RAM, and the like. The CPU reads various process programs stored in the storage section 140, develops the programs in the RAM, and executes various processes in cooperation with the programs. For example, the controller 130 realizes various measurement modes for urine volume, inferior aorta diameter, stool hardness, blood collection guide, and the like by executing a dedicated application(s) 141 stored in the storage section 140.

The controller 130 functions as a communication controller, an acquisition section, and an output section. The functions of the communication controller, the acquisition section, and the output section are implemented by, for example, executing the dedicated application 141 stored in the storage section 140. The communication controller transmits image data based on the ultrasonic image signals obtained by transmission and reception of ultrasonic waves to and from the subject S to the information processing apparatus 20 via the communication part 120. The acquisition section acquires diagnostic information I obtained by the information processing apparatus 20 performing numerical conversion or simplification on three-dimensional image data constructed using two-dimensional image data. The output section outputs the acquired diagnostic information I to the display part 114 or the like via the display controller 112 or the like.

The storage section 140 includes any storage module, which is, for example, an HDD, an SSD, a ROM, a RAM or the like. The storage section 140 stores, for example, a system program, application programs, and various types of data received by the communication part 120. Specifically, the storage section 140 stores the dedicated application 141 for executing various measurement modes.

The ultrasonic probe 150 is a portion that is positioned at the tip side of the housing 100 and is pressed against the body surface of the subject S. The ultrasonic probe 150 includes pMUT elements (transducers) manufactured using, for example, MEMS technology. An example in which the pMUT element(s) is constituted of a plurality of pMUT cells including high-frequency cells and low-frequency cells is cited as a specific aspect. The high-frequency cells and the low-frequency cells each include a resonant frequency and are arranged in a two-dimensional matrix. The MEMS is an abbreviation for Micro Electro Mechanical Systems. The pMUT is an abbreviation for Piezoelectric Micromachined ultrasonic Transducer. For example, in a case where the pMUT element is formed of specific different elements alternately arranged of 384×128, the high-frequency cells and the low-frequency cells of 2×2 may be defined as one channel to configure a matrix transducer of 192×64 channels. The ultrasonic probe 150 transmits ultrasonic waves to the subject S based on a drive signal transmitted from the transmitter 104, and receives reflected waves reflected by the subject S. For example, the ultrasonic probe 150 can perform transmission and reception using 192×1 channels as one slice plane. As an ultrasonic scanning method in the ultrasonic probe 150, for example, a linear scanning method is adopted. Other than the linear scanning method, another scanning method such as a convex scanning method or a sector scanning method may be employed. Further, the number of transducers can be set as appropriate.

### Example Configuration of Information Processing Apparatus 20

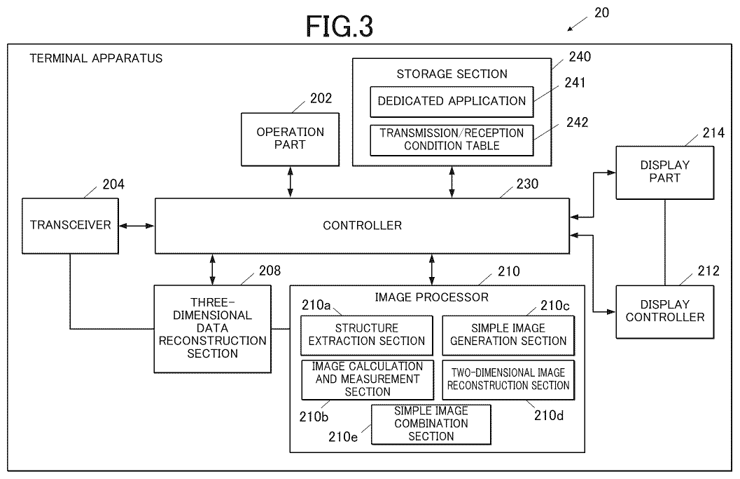

FIG. 3 is a block diagram of the information processing apparatus 20 according to the present embodiment.

The information processing apparatus 20 includes an operation part 202, a transceiver 204, a three-dimensional data reconstruction section 208, an image processor 210, a display controller 212, a display part 214, a controller 230, and a storage section 240.

The operation part 202 receives various input operations from the user, converts the received input operations to electrical signals, and outputs the electrical signals to the controller 230. The operation part 202 includes, for example, a mouse, a keyboard, a trackball, a switch, and a button. The operation part 202 may be, for example, a touch screen integrally combined with a display. The operation part 202 may be, for example, a user interface such as a microphone that receives a voice input.

The transceiver 204 includes, for example, an NIC, a LAN adapter, and a communication module including a receiver and a transmitter. The transceiver 204 communicates various data such as ultrasonic image data, transmission/reception condition information, and diagnostic information I with the ultrasonic diagnostic apparatus 10 or the like connected via the network 30.

The image processor 210 executes predetermined image processing, for example, in accordance with an instruction from the controller 230 or the like. The image processor 210 includes a structure extraction section 210a, an image calculation and measurement section 210b, a simple image generation section 210c, a two-dimensional image reconstruction section 210d, and a simple image combination section 210e. The structure extraction section 210a extracts a specific region such as an echo-free region or a lumen region from three-dimensional data obtained by reconstructing volume data received by the transceiver 204. The image calculation and measurement section 210b calculates and measures the number of voxels or the like in the specific region. The simple image generation section 210c generates a simple image using the diameter, the depth, and the like of the specific region. The two-dimensional image reconstruction section 210d reconstructs a two-dimensional image by selecting a specific section from predetermined volume data. The simple image combination section 210e combines a plurality of simple images generated by the simple image generation section 210c or the like to form one image.

The display controller 212 generates an image signal for display by executing coordinate conversion or the like on the received ultrasonic image data under the control of the controller 230. The display controller 212 outputs the generated image signal for display to the display part 214.

The display part 214 is, for example, a display apparatus such as an LCD or an organic EL display. The display part 214 displays a still image or a moving image corresponding to an image signal for display output from the display controller 212 on the display screen under the control of the controller 230. For example, the display part 214 displays measurement information or the like obtained by numerical conversion or simplification on a three-dimensional image.

The controller 230 includes a processor such as a CPU or a GPU, and a memory such as a RAM. The GPU is an abbreviation for Graphics Processing Unit. The processor reads various process programs stored in the storage section 240, develops the programs in the RAM, and executes various processes in cooperation with the programs. The processor may be formed with a single processor or a plurality of processors. The controller 230 may execute at least part of the functions of the three-dimensional data reconstruction section 208 and the image processor 210. Further, the controller 230 may execute a predetermined process in cooperation with the three-dimensional data reconstruction section 208 and the image processor 210.

The storage section 240 includes any storage module, which is, for example, an HDD, an SSD, a ROM, a RAM or the like. The storage section 240 stores, for example, a system program, application programs, and various types of data received by the communication part 120. The storage section 240 stores a dedicated application(s) 241 for executing each measurement mode, and a transmission/reception condition table 242 that defines transmission/reception conditions of ultrasonic waves corresponding to each measurement mode.

### Configuration Example of Transmission/Reception Condition Table 242

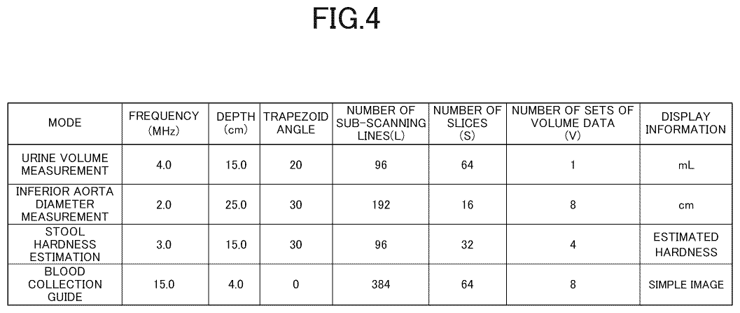

FIG. 4 shows an example of the configuration of the transmission/reception condition table 242 stored in the storage section 240.

In the transmission/reception condition table 242, each measurement mode is associated with transmission/reception conditions of the ultrasonic probe 150 set for each measurement mode. Examples of the measurement mode include a urine volume measurement mode, an inferior aorta diameter measurement mode, a stool hardness measurement mode, and a blood collection guide measurement mode. Examples of items of the transmission/reception conditions of the ultrasonic probe 150 include frequency, echo acquisition depth, trapezoidal angle, the number of sub-scanning lines, the number of slices, the number of sets of volume data, and display information. The frequency is a parameter for adjusting the resolution or the like of an ultrasonic image. The echo acquisition depth is a parameter for adjusting the depth or the like of a measurement portion which is an ultrasonic image. The trapezoid angle is a parameter for adjusting the angle of a trapezoidal portion when ultrasonic waves are emitted in a trapezoidal shape (radially) in a wide-angle scanning method. The number of sub-scanning lines is acoustic line density, and is a parameter indicating, for example, how many reception sound rays are used to draw one fan-shaped slice (scan) plane. The sub-scanning lines can be set without being limited to the transducer element intervals. The number of slices is a parameter for adjusting the amount of image information in the orthogonal direction of a sectional image. The number of volume sets is a parameter indicating the number of sets in the time direction in units of the number of slices specified. The number of volume sets is appropriately set, for example, in a case where a situation change in a time direction such as a fluctuation in diameter of a vein, an artery, or the like is observed. To be specific, for the measurement mode being the urine volume measurement mode, “Frequency: 4 MHz, Echo Acquisition Depth: 15.0 cm, Trapezoidal Angle: 20, Number of Sub-scanning Lines: 96 L, Number of Slices: 64 S, Number of Volume Sets: 1 V, Display Information: mL” are associated with one another and stored.

### [Sequence Diagram of Ultrasonic Diagnostic System 1]

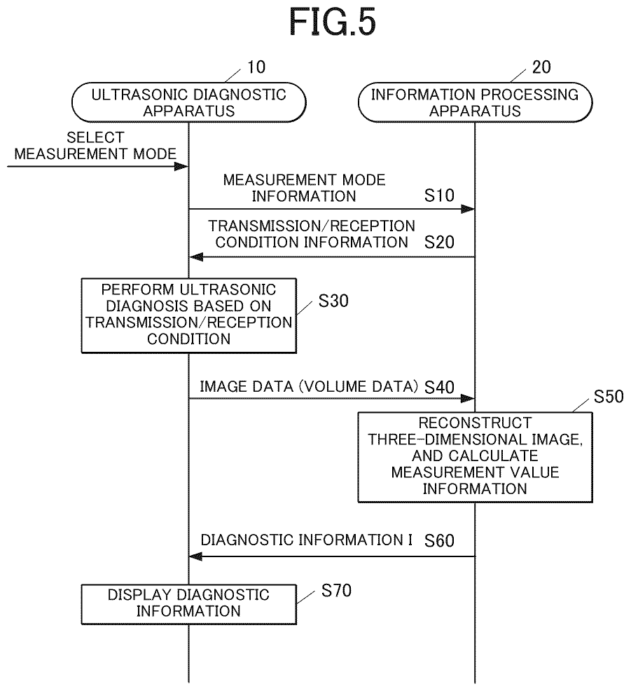

FIG. 5 is a sequence diagram showing a flow of information and data between the ultrasonic diagnostic apparatus 10 and the information processing apparatus 20 constituting the ultrasonic diagnostic system 1.

Upon receiving a user's selection of a predetermined measurement mode, the ultrasonic diagnostic apparatus 10 generates measurement mode information corresponding to the type of the selected measurement mode. The ultrasonic diagnostic apparatus 10 transmits the generated measurement mode information to the information processing apparatus 20 via the network 30 (Step S10).

Upon receiving the predetermined measurement mode information from the ultrasonic diagnostic apparatus 10, the information processing apparatus 20 acquires transmission/reception condition information corresponding to the measurement mode information from the transmission/reception condition table 242. The information processing apparatus 20 transmits the acquired transmission/reception condition information to the ultrasonic diagnostic apparatus 10 via the network 30 (Step S20).

The ultrasonic diagnostic apparatus 10 transmits ultrasonic waves to the subject S on the basis of the transmission/reception conditions received from the information processing apparatus 20. The ultrasonic diagnostic apparatus 10 receives the reflected waves reflected by the subject S, and acquires image data of the bladder, blood vessel(s), intestine(s), and/or the like corresponding to the received reflected waves (Step S30). The ultrasonic diagnostic apparatus 10 sequentially transmits the acquired image data to the information processing apparatus 20 via the network 30 (Step S40).

The information processing apparatus 20 sequentially receives the image data transmitted from the ultrasonic diagnostic apparatus 10. The information processing apparatus 20 reconstructs the three-dimensional image data using the volume data made of the received image data. The information processing apparatus 20 performs a predetermined process on the reconstructed three-dimensional image to numerically convert or simplify the three-dimensional image, thereby generating the diagnostic information I (Step S50). The information processing apparatus 20 transmits the generated diagnostic information I to the ultrasonic diagnostic apparatus 10 via the network 30 (Step S60).

The ultrasonic diagnostic apparatus 10 receives the diagnostic information I transmitted from the information processing apparatus 20. The ultrasonic diagnostic apparatus 10 displays the received diagnostic information I on the screen of the display part 114 (Step S70). The diagnostic information I is, for example, a measured value obtained by numerical conversion on the three-dimensional image data, a simple image obtained by simplification on the three-dimensional image data, or the like.

### Operation Example of Ultrasonic Diagnostic Apparatus 10

Hereinafter, an operation example of the present invention will be described for each mode, but the present invention is not limited to these uses. For example, the urine volume measurement mode can be applied to measurement of ascites accumulated in the abdominal cavity or the amount of bile accumulated in the gallbladder by changing a process parameter value(s). Similarly, the inferior aorta diameter measurement mode can be applied to measurement of the embolization rate due to the plaque of the carotid artery. The stool hardness estimation mode is applicable to follow-up observation of a benign tumor in the liver (presence or absence of a state change), and the blood collection guide mode is applicable not only to blood collection but also to placement of a blood removal and return catheter in dialysis, puncture and placement of an indwelling needle in postoperative anesthesia, placement of a central venous catheter such as a CV port or PICC, arterial puncture called A-line for the purpose of invasive blood pressure measurement, arteriovenous puncture performed in cardiac catheterization, and the like.

### (Urine Volume Measurement Mode)

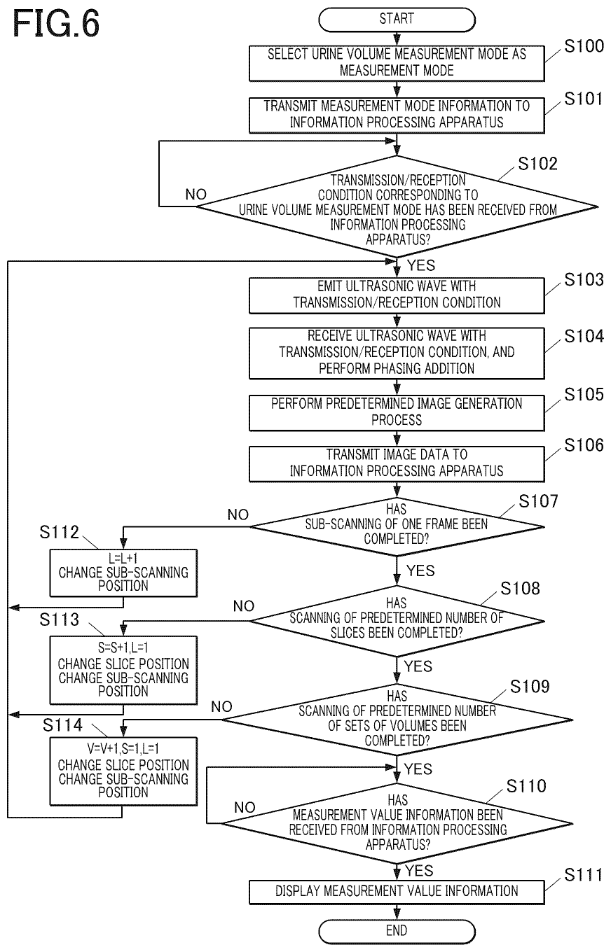

Next, a case where the urine volume measurement mode is selected as the measurement mode will be described. FIG. 6 is a flowchart illustrating an example of the operation of the ultrasonic diagnostic apparatus 10 when the urine volume measurement mode is selected as the measurement mode.

The user selects the urine volume measurement mode as the measurement mode by operating the operation part 102 (Step S100). The controller 130 generates measurement mode information corresponding to the urine volume measurement mode received by the operation part 102.

The controller 130 transmits the generated measurement mode information indicating the urine volume measurement mode to the information processing apparatus 20 via the communication part 120 (Step S101).

The controller 130 determines whether the transmission/reception condition information corresponding to the measurement mode information indicating the urine volume measurement mode transmitted in Step S101 has been received from the information processing apparatus 20 (Step S102). Examples of the transmission/reception condition information for the urine volume measurement mode include a frequency of 4 MHz, an echo acquisition depth of 15.0 cm, a trapezoid angle of 20, the number of sub-scanning lines of 96 L, the number of slices of 64 S, the number of volume sets of 1 V, and display Information of mL. If the controller 130 determines that the communication part 120 has not received the transmission/reception condition information, the controller 130 returns to Step S102. On the other hand, if the controller 130 determines that the communication part 120 has received the transmission/reception condition information, the controller 130 proceeds to Step S103.

The controller 130 controls the ultrasonic probe 150 based on the acquired transmission/reception condition information. The ultrasonic probe 150 transmits ultrasonic waves to the subject S under the control of the controller 130 (Step S103).

The ultrasonic probe 150 receives the reflected waves reflected by the subject S (Step S104). The receiver 106 performs phasing addition on reception signals that are the reflected waves received by the ultrasonic probe 150. The phasing addition circuit of the receiver 106 generates sound ray data (sound ray signals) by phasing addition.

The image generator 108 performs a predetermined image generation process on the sound ray data on which the phasing addition has been performed (Step S105). Examples of the image generation process include amplification, dynamic filtering, envelope detection, and Log compression. The image generator 108 generates image data such as B-mode image data by executing these image generation processes on the sound ray data.

The controller 130 transmits the generated image data to the information processing apparatus 20 via the communication part 120 (Step S106). For example, the controller 130 sequentially transmits, to the information processing apparatus 20, image data of a preset number of sets of volume data as described later, according to the transmission/reception condition information.

The controller 130 determines whether sub-scanning of one frame has been completed (Step S107). If the controller 130 determines that the sub-scanning of one frame has not been completed, the controller 130 proceeds to Step S112. The controller 130 executes L=L+1 to increase a sub-scanning position L by one sub-scanning line, and returns to Step S103 (Step S112). The controller 130 transmits ultrasonic waves to the subject S at the changed sub-scanning position L. On the other hand, if the controller 130 determines that the sub-scanning of one frame has been completed, the controller 130 proceeds to Step S108.

The controller 130 determines whether scanning of a predetermined number of slices has been completed (Step S108). If the controller 130 determines that the scanning of the predetermined number of slices has not been completed, the controller 130 proceeds to Step S113. The controller 130 executes S=S+1 to increase a slice position S by one slice. The controller 130 executes L=1, and transmits ultrasonic waves to the subject S at the first sub-scanning position L of the changed slice position S (Step S113). On the other hand, if the controller 130 determines that the scanning of the predetermined number of slices has been completed, the controller 130 proceeds to Step S109.

The controller 130 determines whether scanning of a predetermined number of sets of volumes has been completed (Step S109). If the controller 130 determines that the scanning of the predetermined number of sets of volumes has not been completed, the controller 130 proceeds to Step S114. The controller 130 executes V=V+1 to increase a volume V by one set. The controller 130 executes S=1 and L=1, and transmits ultrasonic waves to the subject S at the first sub-scanning position L of the first slice position S of the changed volume (Step S114). On the other hand, if the controller 130 determines that the scanning of the predetermined number of sets of volumes has been completed, the controller 130 proceeds to Step S110.

The controller 130 determines whether the diagnostic information I based on the result of processing the image data transmitted in Step S106 has been received from the information processing apparatus 20 (Step S110). The diagnostic information I includes measurement value information corresponding to the amount of urine accumulated in the bladder of the subject S and the like. If the controller 130 determines that the diagnostic information I has not been received from the information processing apparatus 20, the controller 130 returns to Step S110. On the other hand, if the controller 130 determines that the diagnostic information I has been received from the information processing apparatus 20, the controller 130 proceeds to Step S111.

The controller 130 causes the display part 114 to display the diagnostic information I received from the information processing apparatus 20 on the screen of the display part 114 (Step S111). If the controller 130 acquires sectional image data indicating a region including urine of the bladder from the information processing apparatus 20, the controller 130 can cause the display part 114 to display the sectional image data in addition to the diagnostic information I.

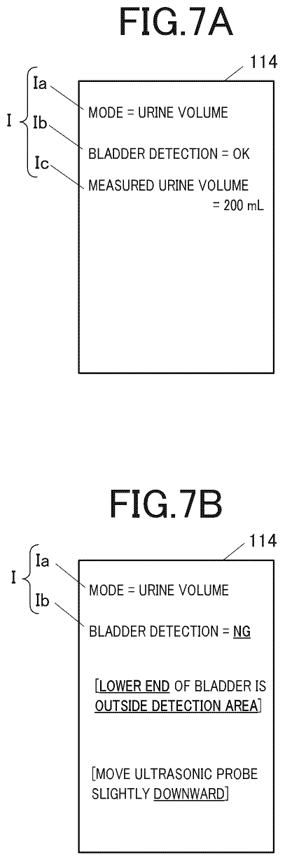

FIG. 7A shows an example of the diagnostic information I displayed on/by the display part 114 when the urine volume measurement mode ends normally. As illustrated in FIG. 7A, mode information Ia indicating the type of the measurement mode, detection information Ib indicating whether the bladder has been detected normally, and measurement value information Ic indicating the amount of urine accumulated in the bladder are displayed as the diagnostic information I on the screen of the display part 114. As the mode information Ia, letters of “Urine Volume” are displayed. As the detection information Ib, letters of “OK” indicating successful detection of the echo-free region of the bladder are displayed. As the measurement value information Ic, a numerical value of “200 mL” is displayed.

FIG. 7B shows an example of the diagnostic information I displayed on/by the display part 114 when the urine volume measurement mode does not end normally. As illustrated in FIG. 7B, mode information Ia and detection information Ib are displayed as the diagnostic information I on the screen of the display part 114. As the mode information Ia, letters of “Urine Volume” are displayed. As the detection information Ib, letters “NG” indicating failed detection of the echo-free region of the bladder are displayed. In addition, in the detection information Ib, for example, letters of “Lower End of Bladder is Outside Detection Area” and “Move Ultrasonic Probe Slightly Downward” are displayed as assistance information for solving the error.

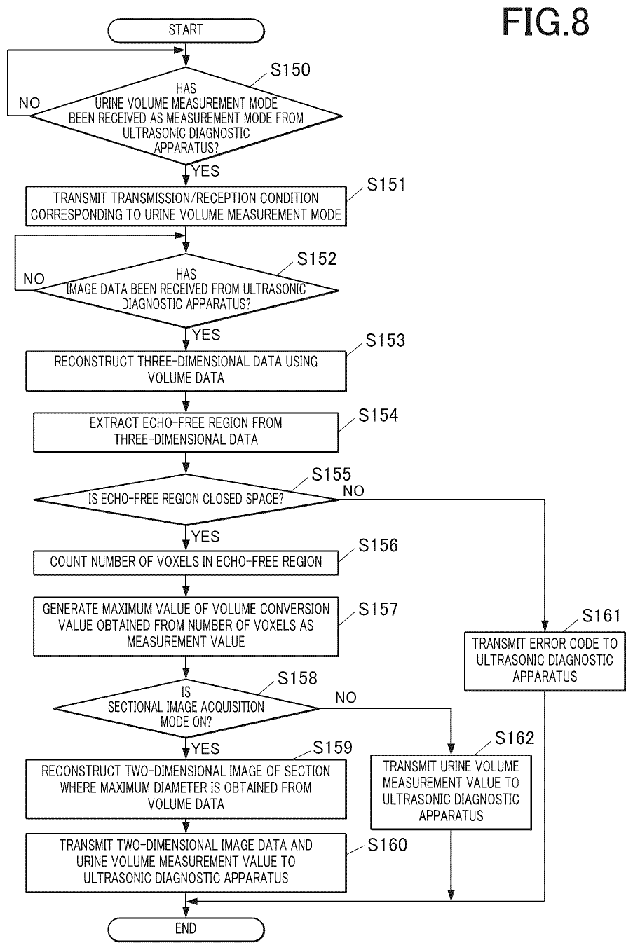

FIG. 8 is a flowchart illustrating an example of the operation of the information processing apparatus 20 when the urine volume measurement mode is selected as the measurement mode.

The controller 230 determines whether measurement mode information indicating the urine volume measurement mode as the measurement mode has been received from the ultrasonic diagnostic apparatus 10 (Step S150). If the controller 230 determines that the measurement mode information has not been received from the ultrasonic diagnostic apparatus 10, the controller 230 returns to Step S150. On the other hand, if the controller 230 determines that the measurement mode information has been received from the ultrasonic diagnostic apparatus 10, the controller 230 proceeds to Step S151.

The controller 230 reads the transmission/reception condition information corresponding to the received urine volume measurement mode from the storage section 240, and transmits the read transmission/reception condition information to the ultrasonic diagnostic apparatus 10 (Step S151). For example, the controller 230 reads, from the transmission/reception condition table 242, as the transmission/reception conditions for the urine volume measurement mode, “Frequency: 4 MHz, Echo Acquisition Depth: 15.0 cm, Trapezoid Angle: 20, Number of Sub-scanning Lines: 96 L, Number of Slices: 64 S, Number of Volume Sets: 1 V, Display Information: mL”.

After transmitting the transmission/reception condition information to the ultrasonic diagnostic apparatus 10, the controller 230 determines whether image data has been received from the ultrasonic diagnostic apparatus 10 (Step S152). If the controller 230 determines that image data of a preset number of sets of volume data has been received, the controller 230 proceeds to Step S153.

The three-dimensional data reconstruction section 208 reconstructs three-dimensional data by using the volume data of the number of sets received (Step S153). For example, the three-dimensional data reconstruction section 208 performs rendering to construct a three-dimensional image of the bladder. The three-dimensional image of the bladder is composed of, for example, voxels.

The image processor 210 extracts the echo-free region from the three-dimensional image including the bladder (Step S154). The echo-free region is, for example, a region indicating urine accumulated in the bladder.

The controller 230 determines whether the echo-free region extracted from the three-dimensional data of the bladder is a closed space (Step S155). Here, the closed space means that the echo-free region does not protrude from the region of the acquired ultrasonic image and the boundary line between the echo-free region and the peripheral tissues is closed. Therefore, in a case where a predetermined amount of urine is accumulated in the bladder, the echo-free region of a closed space appears in the ultrasonic image. If the controller 230 determines that the echo-free region of the three-dimensional data of the bladder is not a closed space, the controller 230 proceeds to Step S161. The controller 230 generates an error code indicating that the border of the echo-free region is not closed, and transmits the diagnostic information I including the generated error code to the ultrasonic diagnostic apparatus 10 (Step S161).

On the other hand, if the controller 230 determines that the echo-free area is a closed space, the controller 230 proceeds to Step S156. The image processor 210 counts the number of voxels in the extracted echo-free region in the bladder (Step S156). Thus, the amount of urine accumulated in the bladder can be estimated from the three-dimensional image data.

The controller 230 generates, as an estimated value, the maximum value of the volume conversion value indicating the urine volume obtained from the number of voxels in the echo-free region (Step S157). The generated estimated value is stored in the storage section 240.

After generating the estimated value corresponding to the amount of urine accumulated in the bladder, the controller 230 determines whether a sectional image acquisition mode is ON (Step S158). In the sectional image acquisition mode, for example, an ultrasonic image at a predetermined slice position obtained by ultrasonic imaging may be used. As the sectional image, an image reconstructed from the acquired three-dimensional image of the bladder may be used. If the controller 230 determines that the sectional image acquisition mode is ON, the controller 230 proceeds to Step S159.

The image processor 210 reconstructs, for example, a two-dimensional image of a section where the maximum size of the echo-free region is obtained, from the volume data received from the ultrasonic diagnostic apparatus 10 (Step S159). The reconstructed two-dimensional sectional image data is stored in the storage section 240.

The controller 230 transmits the two-dimensional sectional image data and the diagnostic information I including the urine volume measurement value to the ultrasonic diagnostic apparatus 10 (Step S160).

On the other hand, if the controller 230 determines that the sectional image acquisition mode is not ON, the controller 230 proceeds to Step S162. The controller 230 transmits the diagnostic information I including the calculated urine volume measurement value to the ultrasonic diagnostic apparatus 10 (Step S162).

According to the urine volume measurement mode, the amount of urine accumulated in the bladder is estimated and quantified (numerically converted) based on the constructed three-dimensional image data of the bladder. Thus, a nurse or the like at the site can accurately and easily grasp how much urine is accumulated in a patient, and the nurse or the like can take appropriate measures for a patient even in telemedicine.

### (Inferior Aorta Diameter Measurement Mode)

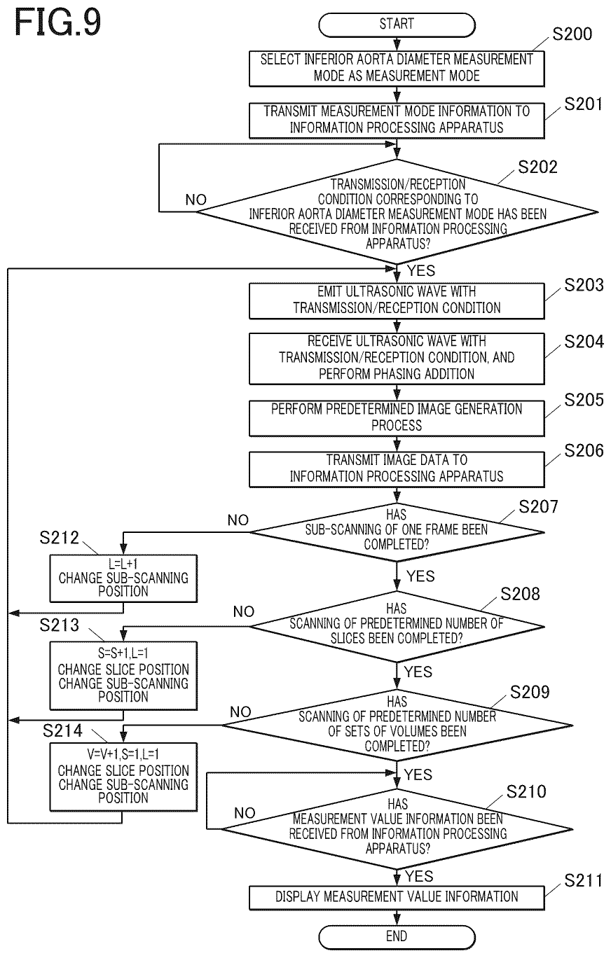

Next, a case where the inferior aorta diameter measurement mode is selected as the measurement mode will be described. FIG. 9 is a flowchart illustrating an example of the operation of the ultrasonic diagnostic apparatus 10 when the inferior aorta diameter measurement mode is selected as the measurement mode.

The user selects the inferior aorta diameter measurement mode as the measurement mode by operating the operation part 102 (Step S200). The controller 130 generates measurement mode information corresponding to the inferior aorta diameter measurement mode received by the operation part 102.

The controller 130 transmits the generated measurement mode information indicating the inferior aorta diameter measurement mode to the information processing apparatus 20 via the communication part 120 (Step S201).

The controller 130 determines whether the transmission/reception condition information corresponding to the measurement mode information indicating the inferior aorta diameter measurement mode transmitted in Step S201 has been received from the information processing apparatus 20 (Step S202). Examples of the transmission/reception condition information for the inferior aorta diameter measurement mode include a frequency of 2 MHz, an echo acquisition depth of 25.0 cm, a trapezoid angle of 30, the number of sub-scanning lines of 192 L, the number of slices of 16 S, the number of volume sets of 8 V, and display information of cm. If the controller 130 determines that the communication part 120 has not received the transmission/reception condition information, the controller 130 returns to Step S202. On the other hand, if the controller 130 determines that the communication part 120 has received the transmission/reception condition information, the controller 130 proceeds to Step S203.

The controller 130 controls the ultrasonic probe 150 and the like based on the acquired transmission/reception condition information. The ultrasonic probe 150 transmits (emits) ultrasonic waves to the subject S under the control of the controller 130 (Step S203).

The ultrasonic probe 150 receives the ultrasonic waves reflected by the subject S (Step S204). The receiver 106 performs phasing addition on the reception signals received by the ultrasonic probe 150. The phasing addition circuit provided in the receiver 106 generates sound ray data (sound ray signals) by phasing addition.

The image generator 108 performs a predetermined image generation process on the sound ray data on which the phasing addition has been performed (Step S205). Examples of the image generation process include amplification, dynamic filtering, envelope detection, and Log compression. The image generator 108 generates image data such as B-mode image data by executing these image generation processes on the sound ray data.

The controller 130 transmits the generated image data to the information processing apparatus 20 via the communication part 120 (Step S206). For example, the controller 130 sequentially transmits, to the information processing apparatus 20, image data of a preset number of sets of volume data as described later, according to the transmission/reception condition information.

The controller 130 determines whether sub-scanning of one frame has been completed (Step S207). If the controller 130 determines that the sub-scanning of one frame has not been completed, the controller 130 proceeds to Step S212. The controller 130 executes L=L+1 to increase the sub-scanning position L by one sub-scanning line, and returns to Step S203 (Step S212). The controller 130 transmits ultrasonic waves to the subject S at the changed sub-scanning position L. On the other hand, if the controller 130 determines that the sub-scanning of one frame has been completed, the controller 130 proceeds to Step S208.

The controller 130 determines whether scanning of a predetermined number of slices has been completed (Step S208). If the controller 130 determines that the scanning of the predetermined number of slices has not been completed, the controller 130 proceeds to Step S213. The controller 130 executes S=S+1 to increase the slice position S by one slice. The controller 130 executes L=1, and transmits ultrasonic waves to the subject S at the first sub-scanning position L of the changed slice position S (Step S213). On the other hand, if the controller 130 determines that the scanning of the predetermined number of slices has been completed, the controller 130 proceeds to Step S209.

The controller 130 determines whether scanning of a predetermined number of sets of volume data has been completed (Step S209). If the controller 130 determines that the scanning of the predetermined number of sets of volume data has not been completed, the controller 130 proceeds to Step S214. The controller 130 executes V=V+1 to increase the volume V by one set. The controller 130 executes S=1 and L=1, and transmits ultrasonic waves to the subject S at the first sub-scanning position L of the first slice position S of the changed volume (Step S214). On the other hand, if the controller 130 determines that the scanning of the predetermined number of sets of volume data has been completed, the controller 130 proceeds to Step S210.

The controller 130 determines whether the diagnostic information I based on the result of processing the image data transmitted in Step S206 has been received from the information processing apparatus 20 (Step S210). The diagnostic information I includes, for example, a measurement value of the diameter of the inferior aorta of the subject S. If the controller 130 determines that the diagnostic information I has not been received from the information processing apparatus 20, the controller 130 returns to Step S210. On the other hand, if the controller 130 determines that the diagnostic information I has been received from the information processing apparatus 20, the controller 130 proceeds to Step S211.

The controller 130 causes the display part 114 to display the diagnostic information I received from the information processing apparatus 20 on the screen of the display part 114 (Step S211).

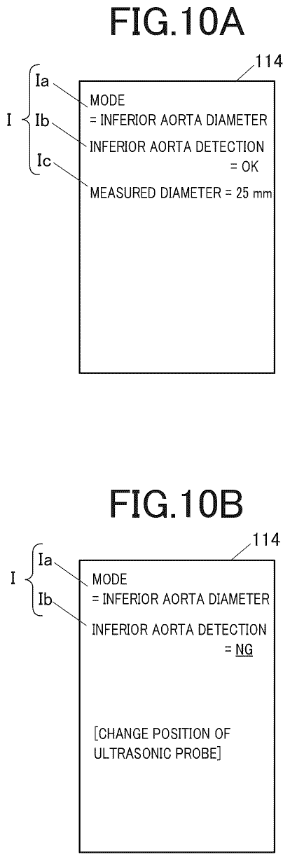

FIG. 10A shows an example of the diagnostic information I displayed on/by the display part 114 when the inferior aorta diameter measurement mode ends normally. As shown in FIG. 10A, mode information Ia indicating the type of the measurement mode, detection information Ib indicating whether the inferior aorta diameter has been detected normally, and measurement value information Ic indicating the diameter of the inferior aorta are displayed as the diagnostic information I on the screen of the display part 114. As the mode information Ia, letters of “Inferior Aorta Diameter” are displayed. As the detection information Ib, letters of “OK” indicating detection of the inferior aorta diameter are displayed. As the measurement value information Ic, a numerical value of “25 mm” is displayed.

FIG. 10B shows an example of the diagnostic information I displayed on/by the display part 114 when the inferior aorta diameter measurement mode does not end normally. As illustrated in FIG. 10B, mode information Ia and detection information Ib are displayed as the diagnostic information I on the screen of the display part 114. As the mode information Ia, letters of “Inferior Aorta Diameter” are displayed. As the detection information Ib, letters of “NG” indicating no detection of the inferior aorta diameter are displayed. In this case, letters of “Change Position of Ultrasonic Probe” are displayed as assistance information for solving the error.

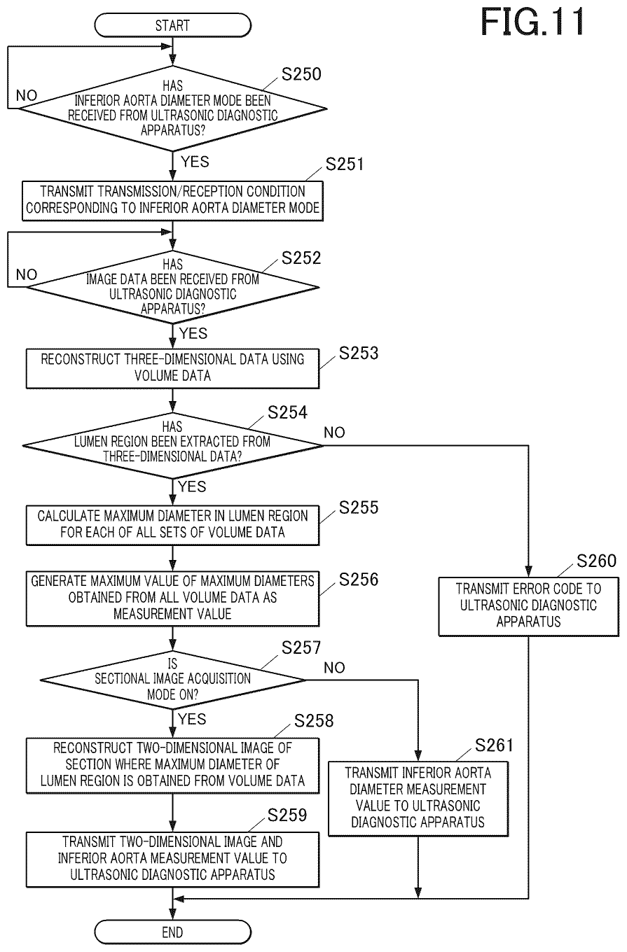

FIG. 11 is a flowchart illustrating an example of the operation of the information processing apparatus 20 when the inferior aorta diameter measurement mode is selected as the measurement mode.

The controller 230 determines whether measurement mode information indicating the inferior aorta diameter measurement mode as the measurement mode has been received from the ultrasonic diagnostic apparatus 10 (Step S250). If the controller 230 determines that the measurement mode information has not been received from the ultrasonic diagnostic apparatus 10, the controller 230 returns to Step S250. On the other hand, if the controller 230 determines that the measurement mode information has been received from the ultrasonic diagnostic apparatus 10, the controller 230 proceeds to Step S251.

The controller 230 reads the transmission/reception condition information corresponding to the received inferior aorta diameter measurement mode from the storage section 240, and transmits the read transmission/reception condition information to the ultrasonic diagnostic apparatus 10 (Step S251). For example, the controller 230 reads, from the transmission/reception condition table 242, as the transmission/reception condition information for the inferior aorta diameter measurement mode, “Frequency: 2 MHz, Echo Acquisition Depth: 25.0 cm, Trapezoid Angle: 30, Number of Sub-scanning Lines: 192 L, Number of Slices: 16 S, Number of Volume Sets: 8 V, Display Information: cm”.

After transmitting the transmission/reception condition information to the ultrasonic diagnostic apparatus 10, the controller 230 determines whether image data has been received from the ultrasonic diagnostic apparatus 10 as a result of execution of ultrasonic diagnosis (Step S252). If the controller 230 determines that image data of a preset number of sets of volume data has been received, the controller 230 proceeds to Step S253.

The three-dimensional data reconstruction section 208 reconstructs three-dimensional data by using the volume data of the number of sets received (Step S253). For example, the three-dimensional data reconstruction section 208 performs rendering to construct three-dimensional image data of the inferior aorta.

The controller 230 determines whether extraction of the lumen region from the three-dimensional image data has succeeded (Step S254). The lumen region includes, for example, the inferior aorta. If the controller 230 determines that the extraction of the lumen region has not succeeded, the controller 230 proceeds to Step S260. For example, this is a case where the position where the ultrasonic probe 150 is pressed against the body surface of the subject S deviates from the inferior aorta. The controller 230 generates an error code indicating failure in the extraction of the lumen region, and transmits the diagnostic information I including the generated error code to the ultrasonic diagnostic apparatus 10 (Step S260).

On the other hand, if the controller 230 determines that the extraction of the lumen region has succeeded, the controller 230 proceeds to Step S255. The controller 230 calculates the maximum diameter in the lumen region in each of all the sets of the volume data (Step S255). For example, by checking the diameter of the inferior aorta, the amount of circulating blood can be grasped, and it can be diagnosed whether heart failure is occurring.

The controller 230 generates, as the measurement value, the maximum value among the maximum diameters obtained from the respective sets of volume data (Step S256). Thus, it can be checked whether the diameter of the inferior aorta having a predetermined length is not off a normal value(s).

After generating the estimated value based on the inferior aorta diameter, the controller 230 determines whether the sectional image acquisition mode is ON (Step S257). If the controller 230 determines that the sectional image acquisition mode is ON, the controller 230 proceeds to Step S258.

The image processor 210 reconstructs a two-dimensional image of a section where the maximum diameter of the inferior aorta is obtained, from the volume data received from the ultrasonic diagnostic apparatus 10 (Step S258). The reconstructed two-dimensional sectional image data is stored in the storage section 240.

The controller 230 transmits the two-dimensional sectional image data and the diagnostic information I including the inferior aorta diameter measurement value illustrated in FIG. 10A or the like to the ultrasonic diagnostic apparatus 10 (Step S259).

On the other hand, if the controller 230 determines that the sectional image acquisition mode is not ON, the controller 230 proceeds to Step S261. The controller 230 transmits the diagnostic information I including the calculated measurement value of the inferior aorta diameter to the ultrasonic diagnostic apparatus 10 (Step S261).

According to the inferior aorta measurement mode, the maximum diameter of the inferior aorta is estimated and quantified (numerically converted) based on the constructed three-dimensional image data of the inferior aorta. In addition, a two-dimensional sectional image of a portion including the maximum diameter of the inferior aorta is generated. Thus, a user such as a nurse at the site can check whether heart failure is not occurring by evaluating whether the inferior aorta diameter is within a certain range, for example, within the range of 12 to 21 mm. Furthermore, the user can appropriately manage the amount of transfusion. As a result, the user can take appropriate measures for the patient even in telemedicine.

### (Stool Hardness Measurement Mode)

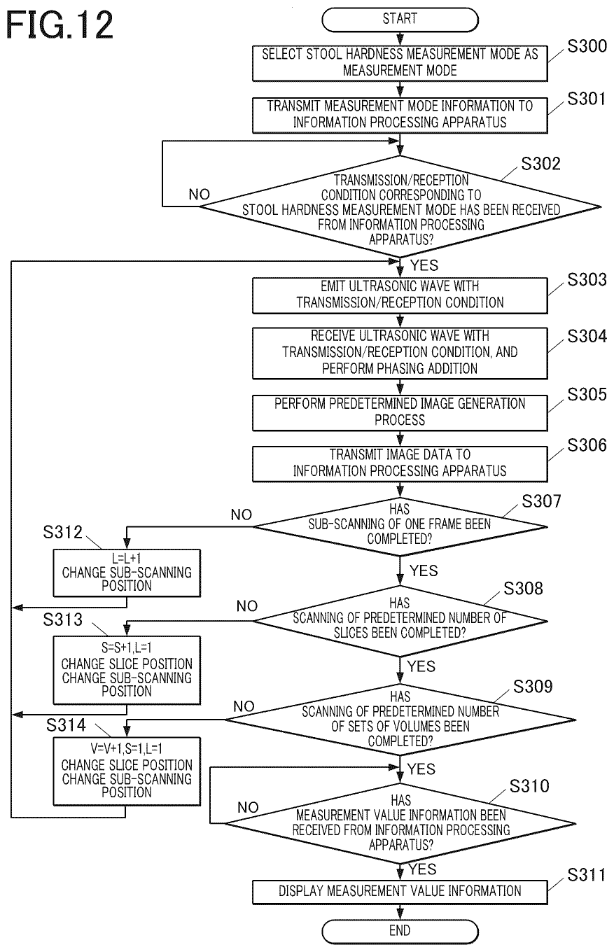

Next, a case where the stool hardness measurement mode is selected as the measurement mode will be described. FIG. 12 is a flowchart showing an example of the operation of the ultrasonic diagnostic apparatus 10 when the stool hardness measurement mode is selected as the measurement mode.

The user selects the stool hardness measurement mode as the measurement mode by operating the operation part 102 (Step S300). The controller 130 generates measurement mode information corresponding to the stool hardness measurement mode received by the operation part 102.

The controller 130 transmits the generated measurement mode information indicating the stool hardness measurement mode to the information processing apparatus 20 via the communication part 120 (Step S301).

The controller 130 determines whether the transmission/reception condition information corresponding to the measurement mode information indicating the stool hardness measurement mode transmitted in Step S301 has been received from the information processing apparatus 20 (Step S302). Examples of the transmission/reception condition information for the stool hardness measurement mode include a frequency of 3 MHz, an echo acquisition depth of 15.0 cm, a trapezoid angle of 30, the number of sub-scanning lines of 96 L, the number of slices of 32 S, the number of volume sets of 4 V, and display information of estimated hardness. If the controller 130 determines that the communication part 120 has not received the transmission/reception condition information, the controller 130 returns to Step S302. On the other hand, if the controller 130 determines that the communication part 120 has received the transmission/reception condition information, the controller 130 proceeds to Step S303.

The controller 130 controls the ultrasonic probe 150 and the like based on the acquired transmission/reception condition information. The ultrasonic probe 150 transmits ultrasonic waves to the subject S under the control of the controller 130 (Step S303).

The ultrasonic probe 150 receives the reflected waves reflected by the subject S (Step S304). The receiver 106 performs phasing addition on reception signals that are the reflected waves received by the ultrasonic probe 150. The phasing addition circuit of the receiver 106 generates sound ray data (sound ray signals) by phasing addition.

The image generator 108 performs a predetermined image generation process on the sound ray data on which the phasing addition has been performed (Step S305). Examples of the image generation process include amplification, dynamic filtering, envelope detection, and Log compression. The image generator 108 generates image data such as B-mode image data by executing these image generation processes on the sound ray data.

The controller 130 transmits the generated image data to the information processing apparatus 20 via the communication part 120 (Step S306). For example, the controller 130 sequentially transmits, to the information processing apparatus 20, image data of a preset number of sets of volume data as described later, according to the transmission/reception condition information.

The controller 130 determines whether sub-scanning of one frame has been completed (Step S307). If the controller 130 determines that the sub-scanning of one frame has not been completed, the controller 130 proceeds to Step S312. The controller 130 executes L=L+1 to increase the sub-scanning position L by one sub-scanning line, and returns to Step S303 (Step S312). The controller 130 transmits ultrasonic waves to the subject S at the changed sub-scanning position L. On the other hand, if the controller 130 determines that the sub-scanning of one frame has been completed, the controller 130 proceeds to Step S308.

The controller 130 determines whether scanning of a predetermined number of slices has been completed (Step S308). If the controller 130 determines that the scanning of the predetermined number of slices has not been completed, the controller 130 proceeds to Step S313. The controller 130 executes S=S+1 to increase the slice position S by one slice. The controller 130 executes L=1, and transmits ultrasonic waves to the subject S at the first sub-scanning position L of the changed slice position S (Step S313). On the other hand, if the controller 130 determines that the scanning of the predetermined number of slices has been completed, the controller 130 proceeds to Step S309.

The controller 130 determines whether scanning of a predetermined number of sets of volume data has been completed (Step S309). If the controller 130 determines that the scanning of the predetermined number of sets of volume data has not been completed, the controller 130 proceeds to Step S314. The controller 130 executes V=V+1 to increase the volume data by one set. The controller 130 executes S=1 and L=1, and transmits ultrasonic waves to the subject S at the first sub-scanning position L of the first slice position S of the changed volume (Step S314). On the other hand, if the controller 130 determines that the scanning of the predetermined number of sets of volume data has been completed, the controller 130 proceeds to Step S310.

The controller 130 determines whether the diagnostic information I based on the result of processing the image data transmitted in Step S306 has been received from the information processing apparatus 20 (Step S310). The diagnostic information I includes, for example, a measurement value indicating the hardness of feces accumulated in the rectum of the subject S. If the controller 130 determines that the diagnostic information I has not been received from the information processing apparatus 20, the controller 130 returns to Step S310. On the other hand, if the controller 130 determines that the diagnostic information I has been received from the information processing apparatus 20, the controller 130 proceeds to Step S311.

The controller 130 causes the display part 114 to display the diagnostic information I received from the information processing apparatus 20 on the screen of the display part 114 (Step S311).

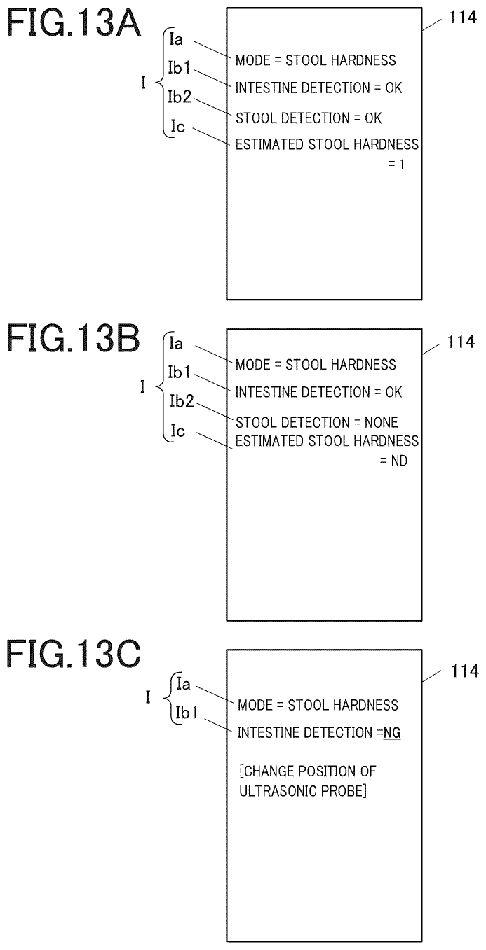

FIG. 13A shows an example of the diagnostic information I displayed on/by the display part 114 when the stool hardness measurement mode has been performed normally. As illustrated in FIG. 13A, mode information Ia indicating the type of the measurement mode, intestine detection information Ib1 indicating whether the rectum has been detected normally, stool detection information Ib2 indicating whether feces in the rectum have been detected, and measurement value information Ic indicating the estimated hardness of the feces are displayed as the diagnostic information I on the screen of the display part 114. As the mode information Ia, letters of “Stool Hardness” are displayed. As the intestine detection information Ib1, letters of “OK” indicating detection of the rectum are displayed. As the stool detection information Ib2, letters of “OK” indicating detection of feces are displayed. As the measurement value information Ic, a numerical value of “1” indicating that the detected feces is hard is displayed. Note that as the index indicating the hardness of feces, for example, the Bristol stool scale, which is a known technology, can be used.

FIG. 13B shows an example of the diagnostic information I displayed on/by the display part 114 when no feces are detected in the stool hardness measurement mode. As illustrated in FIG. 13B, mode information Ia, intestine detection information Ib1, stool detection information Ib2, and measurement value information Ic are displayed as the diagnostic information I on the screen of the display part 114. As the mode information Ia, letters of “Stool Hardness” are displayed. As the intestine detection information Ib1, letters of “OK” indicating detection of the intestine are displayed. As the stool detection information Ib2, letters of “NG” indicating non-detection of feces are displayed. As the measurement value information Ic, a numerical value of “ND” is displayed indicating that the estimation is impossible.

FIG. 13C shows an example of the diagnostic information displayed on/by the display part 114 when the intestine is not detected in the stool hardness measurement mode. As illustrated in FIG. 13C, mode information Ia and intestine detection information Ib1 are displayed as the diagnostic information I on the screen of the display part 114. As the mode information Ia, letters of “Stool Hardness” are displayed. As the intestine detection information Ib1, letters of “NG” indicating no detection of the intestine are displayed. In addition, in the intestine detection information Ib1, for example, letters of “Change Position of Ultrasonic Probe” are displayed as assistance information for solving the error.

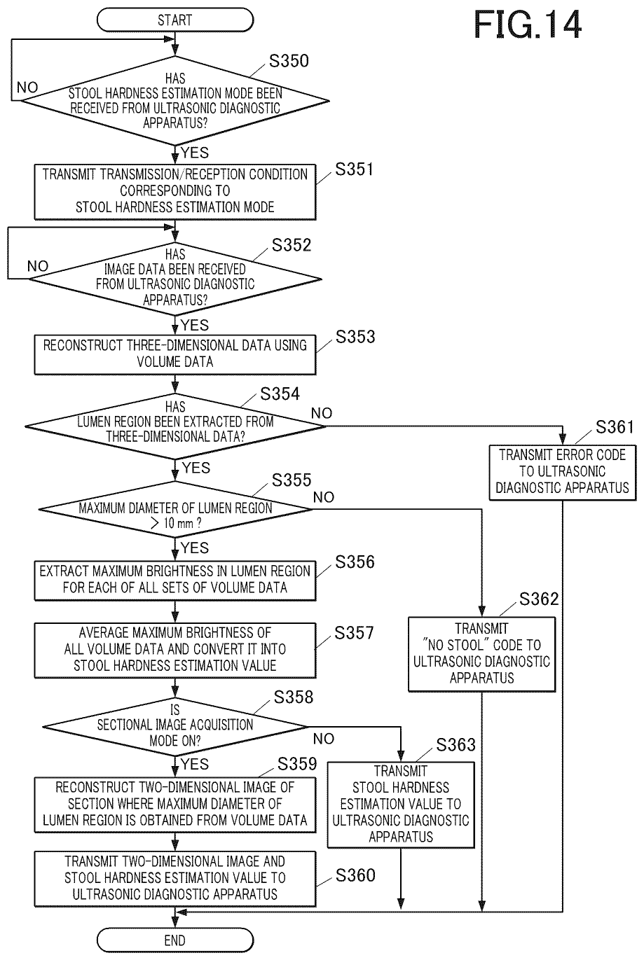

FIG. 14 is a flowchart showing an example of the operation of the information processing apparatus 20 when the stool hardness measurement mode is selected as the measurement mode.

The controller 230 determines whether measurement mode information indicating the stool hardness estimation mode as the measurement mode has been received from the ultrasonic diagnostic apparatus 10 (Step S350). If the controller 230 determines that the measurement mode information has not been received from the ultrasonic diagnostic apparatus 10, the controller 230 returns to Step S350. On the other hand, if the controller 230 determines that the measurement mode information has been received from the ultrasonic diagnostic apparatus 10, the controller 230 proceeds to Step S351.

The controller 230 reads the transmission/reception condition information corresponding to the received stool hardness estimation mode from the storage section 240, and transmits the read transmission/reception condition information to the ultrasonic diagnostic apparatus 10 (Step S351). For example, the controller 230 reads, from the transmission/reception condition table 242, as the transmission/reception condition information for the stool hardness estimation mode, “Frequency: 3 MHz, Echo Acquisition Depth: 15.0 cm, Trapezoid Angle: 30, Number of Sub-scanning Lines: 96 L, Number of Slices: 32 S, Number of Volume Sets: 4 V, Display Information: Estimated Hardness”.

After transmitting the transmission/reception condition information to the ultrasonic diagnostic apparatus 10, the controller 230 determines whether image data has been received from the ultrasonic diagnostic apparatus 10 as a result of execution of ultrasonic diagnosis (Step S352). If the controller 230 determines that image data of a preset number of sets of volume data has been received, the controller 230 proceeds to Step S353.

The three-dimensional data reconstruction section 208 reconstructs three-dimensional data by using the volume data of the number of sets received (Step S353). For example, the three-dimensional data reconstruction section 208 performs rendering to construct three-dimensional image data of the rectum.

The controller 230 determines whether extraction of the lumen region from the three-dimensional image data has succeeded (Step S354). If the controller 230 determines that the extraction of the lumen region has not succeeded, the controller 130 proceeds to Step S361. The controller 230 generates an error code indicating failure in the extraction of the lumen region, and transmits the diagnostic information I including the generated error code to the ultrasonic diagnostic apparatus 10 (Step S361).

On the other hand, if the controller 230 determines that the extraction of the lumen region has succeeded, the controller 230 proceeds to Step S355. The controller 230 determines whether the maximum diameter of the rectum, which is the extracted lumen region, exceeds 10 mm (Step S355). This is because the diameter of the rectum is larger as the amount of feces accumulated in the rectum is larger.

If the controller 230 determines that the maximum diameter of the extracted lumen region does not exceed 10 mm and is equal to or less than 10 mm, the controller 230 determines that no feces are accumulated in the rectum, and proceeds to Step S362. The controller 230 generates a no-feces code indicating that no feces or substantially no feces are present in the rectum, and transmits the diagnostic information I including the generated code to the ultrasonic diagnostic apparatus 10 (Step S362).

On the other hand, if the controller 230 determines that the maximum diameter of the extracted lumen region exceeds 10 mm, the controller 230 proceeds to Step S356. The image processor 210 extracts the maximum brightness in the lumen region about each of all the sets of volume data (Step S356). Thus, the hardness of feces in the rectum can be estimated.

The controller 230 averages the values of the maximum brightness in the extracted lumen region, and converts it into a stool hardness estimation value (Step S357). For example, a table in which the average value of the values of the maximum brightness of all the sets of volume data is associated with the stool hardness estimation value may be prepared in advance, and the table may be stored in the storage section 240. In general, if the stool hardness is high, reflection of ultrasonic waves on the stool surface is strong, so that the brightness on the stool surface is large. Therefore, behind the stool surface, ultrasonic waves do not pass through, so that the brightness is small. On the other hand, if the stool hardness is low, reflection of ultrasonic waves on the stool surface is weak, so that the brightness on the stool surface is small. Therefore, behind the stool surface, ultrasonic waves pass through, so that the brightness is large.

After generating the stool hardness estimation value, the controller 230 determines whether the sectional image acquisition mode is ON (Step S358). If the controller 230 determines that the sectional image acquisition mode is ON, the controller 230 proceeds to Step S359.

The image processor 210 reconstructs a two-dimensional image of a section where the maximum diameter is obtained from the volume data received from the ultrasonic diagnostic apparatus 10 (Step S359). The reconstructed two-dimensional sectional image data is stored in the storage section 240.

The controller 230 transmits the two-dimensional sectional image data and the diagnostic information I including the stool hardness estimation value illustrated in FIG. 13A or the like to the ultrasonic diagnostic apparatus 10 (Step S360).

On the other hand, if the controller 230 determines that the sectional image acquisition mode is not ON, the controller 230 proceeds to Step S363. The controller 230 transmits the diagnostic information I including the calculated stool hardness estimation value to the ultrasonic diagnostic apparatus 10 (Step S363).

According to the stool hardness measurement mode, the hardness of feces accumulated in the rectum is estimated and quantified (numerically converted) based on the constructed three-dimensional image data of the rectum. Thus, a nurse or the like at the site can accurately and easily grasp whether a patient has constipation or diarrhea by checking the numerical value of the stool hardness. As a result, the user such as a nurse can take appropriate measures for a patient even in telemedicine.

### (Blood Collection Guide Mode)

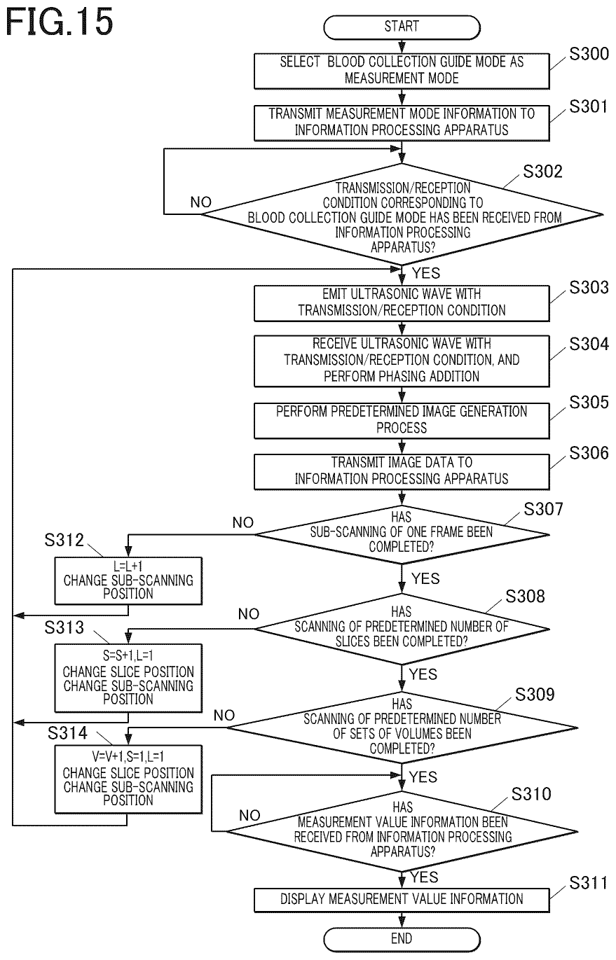

Next, a case where the blood collection guide mode is selected as the measurement mode will be described. FIG. 15 is a flowchart illustrating an example of the operation of the ultrasonic diagnostic apparatus 10 when the blood collection guide mode is selected as the measurement mode.

The user selects the blood collection guide mode as the measurement mode by operating the operation part 102 (Step S400). The controller 130 generates measurement mode information corresponding to the blood collection guide mode received by the operation part 102.

The controller 130 transmits the generated measurement mode information indicating the blood collection guide mode to the information processing apparatus 20 via the communication part 120 (Step S401).

The controller 130 determines whether the transmission/reception condition information corresponding to the measurement mode information indicating the blood collection guide mode transmitted in Step S401 has been received from the information processing apparatus 20 (Step S402). Examples of the transmission/reception condition information for the blood collection guide mode include a frequency of 15 MHz, an echo acquisition depth of 4.0 cm, a trapezoid angle of 0, the number of sub-scanning lines of 384 L, the number of slices of 64 S, the number of volume sets of 8 V, and display information of simple image. If the controller 130 determines that the communication part 120 has not received the transmission/reception condition information, the controller 130 returns to Step S402. On the other hand, if the controller 130 determines that the communication part 120 has received the transmission/reception condition information, the controller 130 proceeds to Step S403.

The controller 130 controls the ultrasonic probe 150 based on the acquired transmission/reception condition information. The ultrasonic probe 150 transmits ultrasonic waves to the subject S under the control of the controller 130 (Step S403).

The ultrasonic probe 150 receives the reflected waves reflected by the subject S (Step S404). The receiver 106 performs phasing addition on reception signals that are the reflected waves received by the ultrasonic probe 150. The phasing addition circuit of the receiver 106 generates sound ray data (sound ray signals) by phasing addition.

The image generator 108 performs a predetermined image generation process on the sound ray data on which the phasing addition has been performed (Step S405). Examples of the image generation process include amplification, dynamic filtering, envelope detection, and Log compression. The image generator 108 generates image data such as B-mode image data by executing these image generation processes on the sound ray data.

The controller 130 transmits the generated image data to the information processing apparatus 20 via the communication part 120 (Step S406). For example, the controller 130 sequentially transmits, to the information processing apparatus 20, image data of a preset number of sets of volume data as described later, according to the transmission/reception condition information.

The controller 130 determines whether sub-scanning of one frame has been completed (Step S407). If the controller 130 determines that the sub-scanning of one frame has not been completed, the controller 130 proceeds to Step S412. The controller 130 executes L=L+1 to increase the sub-scanning position L by one sub-scanning line, and returns to Step S403 (Step S412). The controller 130 transmits ultrasonic waves to the subject S at the changed sub-scanning position L. On the other hand, if the controller 130 determines that the sub-scanning of one frame has been completed, the controller 130 proceeds to Step S408.

The controller 130 determines whether scanning of a predetermined number of slices has been completed (Step S408). If the controller 130 determines that the scanning of the predetermined number of slices has not been completed, the controller 130 proceeds to Step S413. The controller 130 executes S=S+1 to increase the slice position S by one slice. The controller 130 executes L=1, and transmits ultrasonic waves to the subject S at the first sub-scanning position L of the changed slice position S (Step S413). On the other hand, if the controller 130 determines that the scanning of the predetermined number of slices has been completed, the controller 130 proceeds to Step S409.

The controller 130 determines whether scanning of a predetermined number of sets of volumes has been completed (Step S409). If the controller 130 determines that the scanning of the predetermined number of sets of volumes has not been completed, the controller 130 proceeds to Step S414. The controller 130 executes V=V+1 to increase the volume V by one set. The controller 130 executes S=1 and L=1, and transmits ultrasonic waves to the subject S at the first sub-scanning position L of the first slice position S of the changed volume (Step S414). On the other hand, if the controller 130 determines that the scanning of the predetermined number of sets of volumes has been completed, the controller 130 proceeds to Step S410.

The controller 130 determines whether the diagnostic information I based on the result of processing the image data transmitted in Step S406 has been received from the information processing apparatus 20 (Step S410). The diagnostic information I includes a simple image or the like obtained by simplifying the three-dimensional image data including a blood vessel and a nerve. If the controller 130 determines that the diagnostic information I has not been received from the information processing apparatus 20, the controller 130 returns to Step S410. On the other hand, if the controller 130 determines that the diagnostic information I has been received from the information processing apparatus 20, the controller 130 proceeds to Step S411.

The controller 130 causes the display part 114 to display the diagnostic information I including the simple image in which the positions, depths, and the like of a nerve Ne, a vein Ve, and an artery Ar transmitted from the information processing apparatus 20 are simply drawn on the screen of the display part 114 (Step S411).

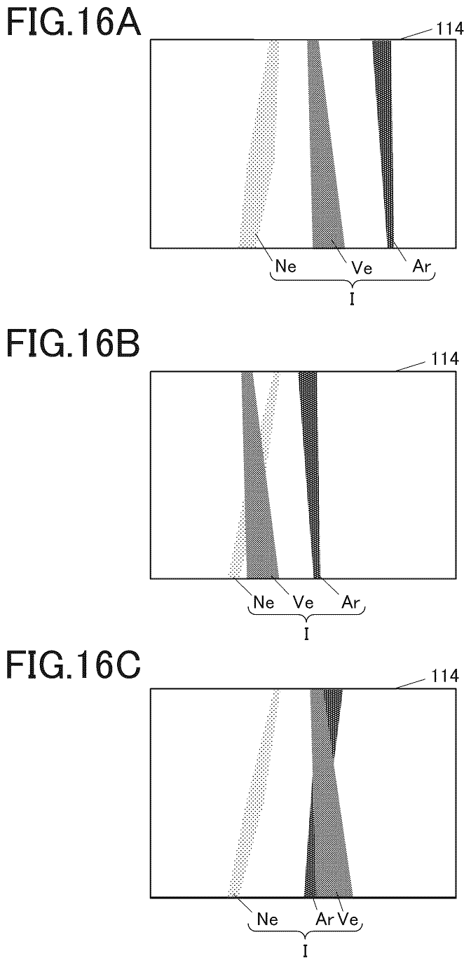

FIGS. 16A to 16C show examples of the diagnostic information I displayed on/by the display part 114 when the blood collection guide mode ends normally. As illustrated in FIGS. 16A to 16C, the simplified nerve Ne, vein Ve, and artery Ar are displayed as the diagnostic information I on the screen of the display part 114. Thus, when a user such as a nurse administers an injection into the vein Ve of a patient, the user can clearly check the positional relationship between the vein Ve and the nerve Ne, the artery Ar, and the like.

In the example shown in FIG. 16A, the nerve Ne, the vein Ve, and the artery Ar are positioned at predetermined intervals, and there is nothing to be avoided near the vein Ve.

In the example shown in FIG. 16B, the vein Ve and the nerve Ne intersect three-dimensionally, and the nerve Ne to be avoided is positioned near the vein Ve.

In the example shown in FIG. 16C, the vein Ve and the artery Ar intersect three-dimensionally, and the artery Ar to be avoided is positioned near the vein Ve.

Therefore, in the cases of FIGS. 16B and 16C, the user needs to administer an injection into the vein Ve while paying attention to the nerve Ne and the artery Ar.

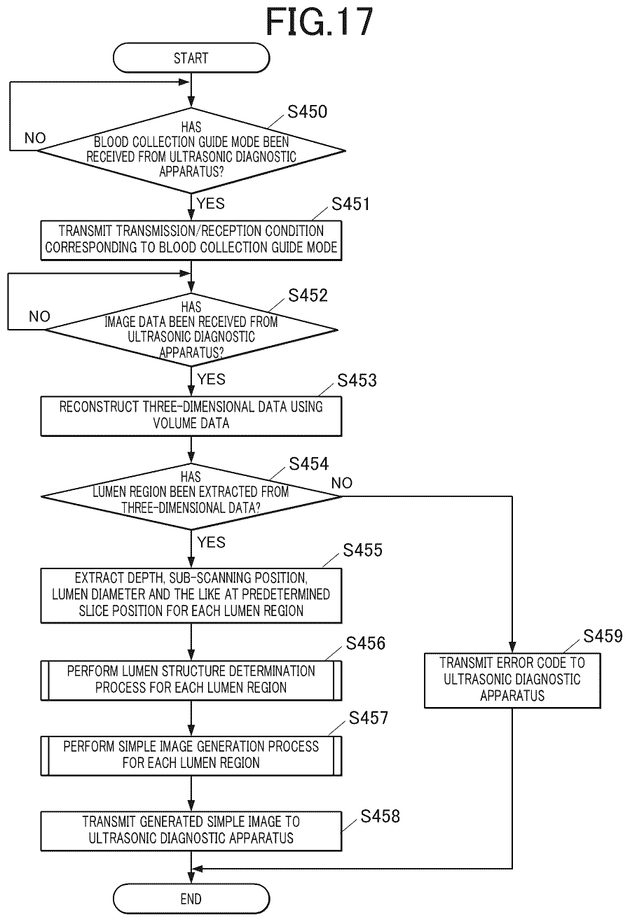

FIG. 17 is a flowchart illustrating an example of the operation of the information processing apparatus 20 when the blood collection guide mode is selected as the measurement mode.

The controller 230 determines whether measurement mode information indicating the blood collection guide mode as the measurement mode has been received from the ultrasonic diagnostic apparatus 10 (Step S450). If the controller 230 determines that the measurement mode information has not been received from the ultrasonic diagnostic apparatus 10, the controller 230 returns to Step S450. On the other hand, if the controller 230 determines that the measurement mode information has been received from the ultrasonic diagnostic apparatus 10, the controller 230 proceeds to Step S451.

The controller 230 reads the transmission/reception condition information corresponding to the received blood collection guide mode from the storage section 240, and transmits the read transmission/reception condition information to the ultrasonic diagnostic apparatus 10 (Step S451). For example, the controller 230 reads, from the transmission/reception condition table 242, as the transmission/reception condition information for the blood collection guide mode, “Frequency: 15 MHz, Echo Acquisition Depth: 4.0 cm, Trapezoid Angle: 0, Number of Sub-scanning Lines: 384 L, Number of Slices: 64 S, Number of Volume Sets: 8 V, Display Information: Simple Image”.

After transmitting the transmission/reception condition information to the ultrasonic diagnostic apparatus 10, the controller 230 determines whether image data has been received from the ultrasonic diagnostic apparatus 10 (Step S452). If the controller 230 determines that image data of a preset number of sets of volume data has been received, the controller 230 proceeds to Step S453.

The three-dimensional data reconstruction section 208 reconstructs three-dimensional data by using the volume data of the number of sets received (Step S453). For example, the three-dimensional data reconstruction section 208 performs rendering to construct a three-dimensional image of the lumen region including a blood vessel, a nerve, and/or the like.

The controller 230 determines whether extraction of the lumen region from the three-dimensional image data has succeeded (Step S454). If the controller 230 determines that the extraction of the lumen region has not succeeded, the controller 230 proceeds to Step S459. The controller 230 generates an error code indicating that the lumen region is not present in the three-dimensional image data, and transmits the diagnostic information I including the generated error code to the ultrasonic diagnostic apparatus 10 (Step S459).

On the other hand, if the controller 230 determines that the extraction of the lumen region has succeeded, the controller 230 proceeds to Step S455. The image processor 210 extracts, for each extracted lumen region, the depth, the sub-scanning position, the lumen diameter and the like at a predetermined slice position (Step S455). Specifically, the image processor 210 extracts a slice position SM where the depth is the shallowest, and extracts a depth DM, a lumen structure central sub-scanning position AM and a lumen diameter RM at the slice position SM where the depth is the shallowest. The reason why the shallowest portion is used is that if there is no nerve or the like to be avoided in the periphery, the insertion depth can be the shallowest, and since the insertion depth is shallow, a problem that a needle does not go straight during the insertion or the like hardly occurs. The image processor 210 extracts a depth DF, a lumen structure central sub-scanning position AF, and a lumen diameter RF at the most distal slice position. The image processor 210 extracts a depth DN, a lumen structure central sub-scanning position AN, and a lumen diameter RN at the most proximal slice position.

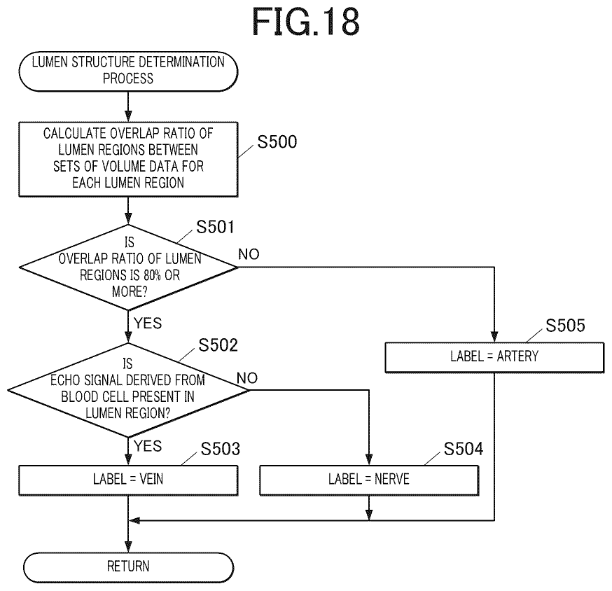

Next, a lumen structure determination process is performed on each extracted lumen region. The lumen structure determination process is executed by a subroutine shown in FIG. 18.

FIG. 18 is a flowchart illustrating the lumen structure determination process.

The controller 230 calculates the overlap ratio of the lumen regions between the sets of volume data at the slice position SM extracted for each lumen region (Step S500). Here, for example, since the diameter of an artery changes on the time axis in accordance with the heartbeat, the overlap ratio of the lumen regions decreases. On the other hand, since the diameters of an artery and a nerve do not change on the time axis in accordance with the heartbeat, the overlap ratio of the lumen regions does not decrease. In the present embodiment, an artery is distinguished from the others, that is, a vein and a nerve, by calculating the overlap ratio of the lumen regions.

The controller 230 determines whether the calculated overlap ratio of the lumen regions is 80% or more (Step S501). If the controller 230 determines that the calculated overlap ratio of the lumen regions is 80% or more, the controller 230 proceeds to Step S502. In this case, it can be determined that the lumen structure is a vein or a nerve.

The controller 230 determines whether there is an echo signal derived from blood cells in the lumen region (Step S502). In veins, blood cells such as red blood cells flow. Therefore, the controller 230 can make a distinction between a vein and a nerve by capturing an echo signal derived from blood cells. If the controller 230 determines that there is an echo signal derived from blood cells in the lumen region, the controller 230 proceeds to Step S503. The controller 230 determines that a predetermined lumen structure is a vein, and attaches a vein label to the predetermined lumen structure (Step S503).

On the other hand, if the controller 230 determines that there is no echo signal derived from blood cells in the lumen region, the controller 230 proceeds to Step S504. The controller 230 determines that the predetermined lumen structure is a nerve, and attaches a nerve label to the predetermined lumen structure (Step S504).

If the controller 230 determines in Step S501 that the overlap ratio of the lumen regions is less than 80%, the controller 230 proceeds to Step S505. The controller 230 determines that the predetermined lumen structure is an artery, and attaches an artery label to the predetermined lumen structure (Step S505). This is because regarding arteries, the overlap ratio of the lumen regions decreases to less than 80% due to change in the diameter due to the heartbeat.

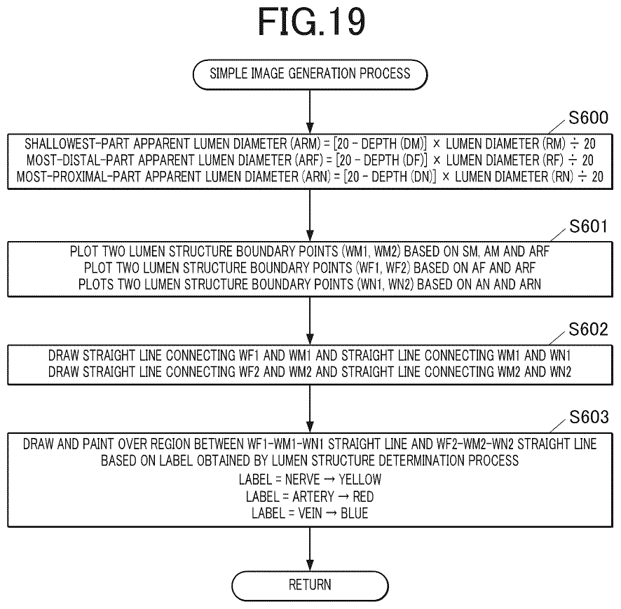

When the subroutine of the lumen structure determination process ends, the controller 230 proceeds to Step S457 shown in FIG. 17. Subsequently, a simple image generation process is performed on each lumen region (Step S457). The simple image generation process is executed by a subroutine illustrated in FIG. 19.

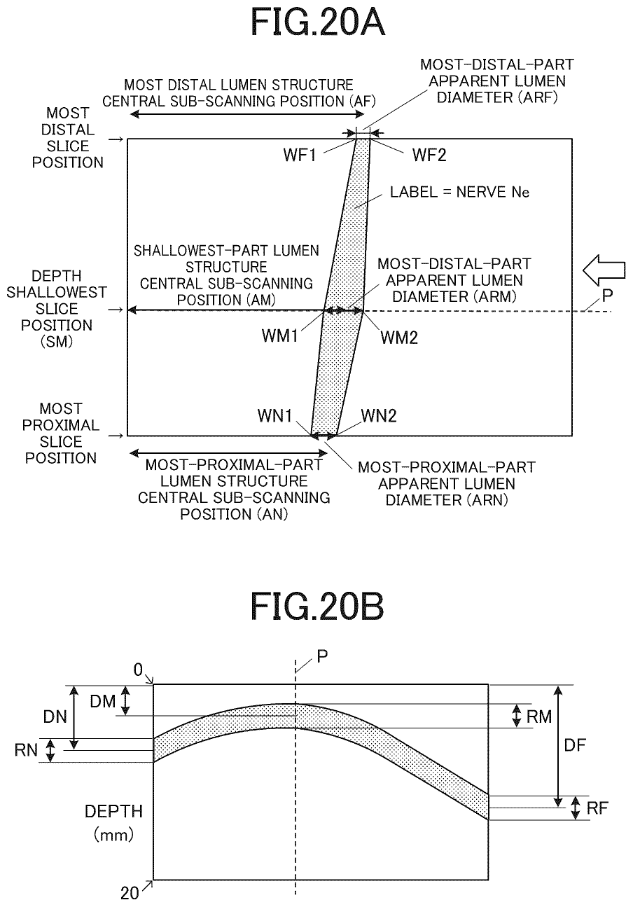

FIG. 19 is a flowchart illustrating an example of the simple image generation process. FIGS. 20A and 20B are diagrams for explaining a procedure for generating a simple image of the nerve Ne as the lumen region. Specifically, FIG. 20A shows the lumen region as viewed from above the skin, and FIG. 20B shows the lumen region as viewed in the depth direction (direction indicated by the arrow in FIG. 20A). In FIG. 20A and FIG. 20B, a plane intersecting the slice position SM where the depth is the shallowest is defined as a plane P. The constant value of 20 in FIG. 19 is an example of the depth of the region of interest when an upper limb is punctured. If this function is applied to a lower limb or the neck, appropriate changes are made, for example, the constant value is set to 40.

The controller 230 calculates an apparent lumen diameter for each lumen region in order to draw the vein Ve, the artery Ar, the nerve Ne, and/or the like in a perspective manner according to the depth (Step S600). To be specific, as illustrated in FIGS. 20A and 20B, the controller 230 calculates the shallowest-part apparent lumen diameter ARM by the following formula.

Shallowest-Part Apparent Lumen Diameter ARM=(20−Depth DM)×Lumen Diameter RM÷20

The controller 230 calculates the most-distal-part apparent lumen diameter ARF by the following formula.

Most-Distal-Part Apparent Lumen Diameter ARF=(20−Depth DF)×Lumen Diameter RF÷20

The controller 230 calculates the most-proximal-part apparent lumen diameter ARN by the following formula.

Most-Proximal-Part Apparent Lumen Diameter ARN=(20− Depth DN)×Lumen Diameter RN÷20

The image processor 210 specifies and plots the boundary of the lumen region using the calculated apparent lumen diameters and the like (Step S601). To be specific, as shown in FIG. 20A, the image processor 210 plots two boundary points WM1 and WM2 indicating the lumen region on the basis of the shallowest-part apparent lumen diameter ARM, the most-distal-part apparent lumen diameter ARF, and the most-proximal-part apparent lumen diameter ARN. The image processor 210 plots two boundary points WF1 and WF2 indicating the lumen region on the basis of the lumen structure central sub-scanning position AF and the most-distal-part apparent lumen diameter ARE. The image processor 210 plots two boundary points WN1 and WN2 indicating the lumen region on the basis of the lumen structure central sub-scanning position AN and the most-proximal-part apparent lumen diameter ARN.

The image processor 210 draws each lumen region on the basis of the plotted boundary points (Step S602). To be specific, the image processor 210 draws a straight line connecting the boundary points WF1 and WM1 and a straight line connecting the plotted boundary points WM1 and WN1. The image processor 210 draws a straight line connecting the plotted boundary points WF2 and WM2 and a straight line connecting the plotted boundary points WM2 and WN2. Thus, even in the case of the lumen structure having substantially the same diameter, a simple image in which drawing in a perspective manner has been done according to the depth can be generated. Note that although the case of drawing a simple image of the nerve Ne has been described with reference to FIGS. 20A and 20B, simple images of the vein Ve and the artery Ar can be drawn by the same procedure as that for the nerve Ne.

The image processor 210 draws each drawn lumen region in colors corresponding to the respective types of the nerve Ne, the artery Ar and the vein Ve labeled on the basis of the lumen structure determination result (Step S603). To be more specific, as shown in FIG. 20A, in the case where a nerve label is attached, the image processor 210 paints over a region surrounded by a straight line connecting the boundary points WF1, WM1 and WN1 and a straight line connecting the boundary points WF2, WM2 and WN2, for example, in yellow. Although not illustrated, in a case where an artery is attached, the image processor 210 paints over a region surrounded by boundary points, for example, in red. Although not illustrated, in a case where a vein label is attached, the image processor 210 paints over a region surrounded by boundary points, for example, in blue. The image processor 210 generates the diagnostic information I including a simple image in which all the drawn nerve Ne, artery Ar and vein Ve are superimposed (combined) shown in FIG. 16A or the like. Upon completion of Step S604, the controller 130 proceeds to Step S458 shown in FIG. 17.

The controller 230 transmits the diagnostic information I including the simple image generated by the image processor 210 to the ultrasonic diagnostic apparatus 10 (Step S458).

According to the blood collection guide mode, a simple image in which the positions of a blood vessel(s), a nerve(s) and/or the like are simplified is generated based on the constructed three-dimensional image data of the blood vessel, the nerve and/or the like. Thus, a nurse or the like at the site can accurately and easily grasp the positional relationship between, of a patient, the vein Ve into which an injection is administered and the other, such as the nerve Ne and the artery Ar. As a result, a nurse or the like can take appropriate measures for a patient even in telemedicine.

As described above, according to the present embodiment, the ultrasonic diagnostic apparatus 10 does not handle three-dimensional image data, and the external information processing apparatus 20 processes the three-dimensional image data. Thus, since the ultrasonic diagnostic apparatus 10 does not require a high-speed processor or the like, it is possible to provide the ultrasonic diagnostic apparatus 10 that is compact and easy to carry. Further, according to the ultrasonic diagnostic apparatus 10 of the present embodiment, since a high-speed processor or the like is not required, it is possible to reduce a load, to increase the speed of processing, and to reduce power consumption. Further, since the ultrasonic diagnostic apparatus 10 merely transmits image data of one frame to the information processing apparatus 20 sequentially, no complicated operation is necessary, and heat generation from the controller 130 or the like including a CPU can be suppressed.

Further, conventionally, at the site of telemedicine, it is necessary to make a diagnosis on a patient with a tomographic image including complicated tissues or the like, which is difficult for a user such as a nurse to understand the tomographic image. In contrast, according to the present embodiment, the diagnostic information I obtained by the information processing apparatus 20 performing numerical conversion or simplification on the three-dimensional image data is displayed on the screen of the display part 114 of the ultrasonic diagnostic apparatus 10. Thus, even a nurse or the like can easily understand the result of ultrasonic diagnosis, and can take appropriate and speedy measures for a patient.

Although one or more preferred embodiments of the present disclosure have been described and illustrated in detail with reference to the accompanying drawings, the technical scope of the present disclosure is not limited to such examples. The disclosed embodiments are made for purposes of illustration and example only and not limitation. The scope of the present invention should be interpreted by terms of the appended claims. Those having ordinary knowledge in the technical field of the present disclosure will naturally understand that various modifications and improvements within the scope of the technical idea described in claims are within the technical scope of the present disclosure.

The entire disclosure of Japanese Patent Application No. 2023-195591, filed on Nov. 17, 2023, including description, claims, drawings and abstract is incorporated herein by reference.

What is claimed is:

1. An ultrasonic diagnostic apparatus comprising:
an ultrasonic probe that transmits an ultrasonic wave to a subject and receives an ultrasonic image signal reflected in the subject;
a communicator that transmits image data based on the ultrasonic image signal of the subject received by the ultrasonic probe to an information processing apparatus;
a hardware processor that acquires diagnostic information obtained by the information processing apparatus constructing, using the image data, three-dimensional image data and performing a predetermined process on the three-dimensional image data; and
a display that displays the acquired diagnostic information.

2. The ultrasonic diagnostic apparatus according to claim 1, wherein the predetermined process is a process of numerically converting the three-dimensional image data or a process of simplifying the three-dimensional image data.

3. The ultrasonic diagnostic apparatus according to claim 1, wherein the ultrasonic probe, the communicator and the hardware processor are provided in a same housing.

4. The ultrasonic diagnostic apparatus according to claim 1, wherein the ultrasonic probe includes a plurality of elements having a micro electro mechanical systems (MEMS) structure and arranged two-dimensionally.

5. The ultrasonic diagnostic apparatus according to claim 1, wherein the information processing apparatus is a smartphone, a tablet, a general-purpose personal computer or a workstation.

6. The ultrasonic diagnostic apparatus according to claim 1, wherein the display includes a liquid crystal display or an organic EL display that is a flat panel or a bendable panel.

7. An ultrasonic diagnostic system comprising:
an ultrasonic diagnostic apparatus that transmits an ultrasonic wave to a subject and acquires image data corresponding to an ultrasonic image signal reflected in the subject, and transmits the image data; and
an information processing apparatus that constructs, using the image data transmitted from the ultrasonic diagnostic apparatus, three-dimensional image data, acquires diagnostic information by performing a predetermined process on the three-dimensional image data, and transmits the diagnostic information,
wherein the ultrasonic diagnostic apparatus receives the diagnostic information transmitted from the information processing apparatus, and displays the diagnostic information on a display thereof.

8. The ultrasonic diagnostic system according to claim 7,
wherein the image data includes a bladder, an intestine or a blood vessel, and
wherein the information processing apparatus performs, as the predetermined process, quantification of an amount of urine accumulated in the bladder, quantification of a hardness of feces accumulated in the intestine or quantification of a diameter of the blood vessel.

9. The ultrasonic diagnostic system according to claim 7,
wherein the image data includes at least a blood vessel and a nerve, and
wherein the information processing apparatus performs, as the predetermined process, generation of a simple image indicating arrangement of one or both of the blood vessel and the nerve.

10. A non-transitory computer-readable storage medium storing a program causing a computer to perform:
transmitting, to an information processing apparatus, image data based on an ultrasonic image signal obtained by transmission and reception of an ultrasonic wave to and from a subject;
acquiring diagnostic information obtained by the information processing apparatus constructing, using the image data, three-dimensional image data and performing a predetermined process on the three-dimensional image data; and
outputting the acquired diagnostic information.
